(19) United States
(12) Patent Application Publication
(10) Pub. No.: US 2017/0067882 A1
(43) Pub. Date: Mar. 9, 2017

(54) PHYSIOLOGICALLY-RELEVANT AFFINITY MEASUREMENTS IN VITRO WITH BACKSCATTERING INTERFEROMETRY

(71) Applicants: VANDERBILT UNIVERSITY, Nashville, TN (US); PFIZER INC., New York, NY (US)

(72) Inventors: Darryl J. BORNHOP, Nashville, TN (US); Amanda KUSSROW, Nashville, TN (US); Denise M. O'HARA, Reading, MA (US); Mengmeng WANG, Andover, MA (US)

(21) Appl. No.: 15/120,448

(22) PCT Filed: Feb. 20, 2015
(86) PCT No.: PCT/US15/16944

§ 371 Date: Aug. 19, 2016

Related U.S. Application Data
(60) Provisional application No. 61/942,251, filed on Feb. 20, 2014.

(51) Int. Cl. G01N 33/53; G01N 21/51; G01N 21/45

(52) U.S. Cl. CPC G01N 33/5302; G01N 21/45; G01N 21/51

(57) ABSTRACT

Disclosed herein are improved optical detection methods comprising interferometric detection systems and methods of detecting a binding interaction between a sample comprising uncultured tissue homogenate and an analyte, together with various applications of the disclosed techniques. This abstract is intended as a scanning tool for purposes of searching in the particular art and is not intended to be limiting of the present invention.

### CROSS-REFERENCE TO RELATED APPLICATIONS

This Application claims the benefit of U.S. Provisional Application No. 61/942,251, filed on Feb. 20, 2014, which is incorporated herein by reference in its entirety.

### BACKGROUND

Lack of correlation between in vitro binding/potency and in vivo pharmacological activity and lack of translation of efficacy and safety from preclinical models to human physiology are the two biggest challenges in pharmaceutical development (Kola, I, Landis J. (2004) Nat Rev Drug Discov. 3, 711-715; Peck, R. W. (2007) Drug Discov. Today 12, 289-294; Sultana, S. R., et al. (2007) Drug Discov. Today 12, 419-425). It is often hoped that the in vitro binding affinity or potency (i.e., K_{d} or IC₅₀) can be used to predict the in vivo pharmacological activity (EC₅₀), thus facilitating more accurate translation across species to aid in clinical dose selection and efficacy prediction. Establishment of a correlation between in vitro potency and in vivo activity is crucial for validation of the target enzyme and for achieving confidence in an in vitro screening strategy.

Based on pharmacokinetic/pharmacodynamic (PK-PD) modeling and simulation practice, the K_{d} value is recognized as being key to robust prediction of target coverage and human dose projections for first in human clinical studies (Agoram, B. M., et al. (2007) Drug Discov. Today 12, 1018-1024). In order to prevent over- or under-estimating pharmacology and human dose prediction, it is essential that in vitro Kₐ measurements be representative of the physiological setting. However, no in vitro method capable of confidently establishing in vitro in vivo correlation (IVIVC) has been reported.

It has been hypothesized that the average free efficacious concentration at steady-state in vivo should correlate with the intrinsic (un-bound) potency determined from an in vitro assay (DeGuchi, Y., et al. (1992) J. Pharmacobiodyn. 15, 79-89; Wagner, J. G. (1976) Eur. J. Clin. Pharmacol. 10, 425-432; Wright, J. D., et al. (1996) Clin. Pharmacokinet. 30, 445-462). In practice, however, this relationship is often obscured or confounded by a variety of factors that occur in in vitro assays. These factors include: 1) the inability to reproduce the physiological state of the biotherapeutic drug interacting with the protein target (i.e., non-native expression levels of the protein target may be used, labeling the biotherapeutic drug with chemical entities may be necessary to visualize binding, the extracellular membrane-bound target protein may be soluble and able to be expressed and purified, and/or the biotherapeutic or target protein may be immobilized on a solid surface); 2) non-physiological environments are often used in vitro that do not represent specific and non-specific interactions with biological matrix components and any topology difference due to co-associated proteins; and 3) complex pharmacokinetic/pharmacodynamic relationships may arise due to indirect effects or target site disequilibrium, especially at diseased states. These factors are further illustrated in FIGS. 1A and 1B.

Current technologies widely used to quantify molecular interactions include cell-based binding assays for membrane-bound target proteins that require fluorescence or radioisotope labeling of the biotherapeutic or secondary labeled reagents and sometimes highly expressed membrane-bound targets, and plate- or chip-based assays for soluble target proteins that require one partner of the interaction to be immobilized. Due to the addition of labels, immobilization, buffer environment or washing steps, current methods often do not reflect the K_{d} value observed in physiological conditions. A recently developed label-free, mix-and-read technology, back-scattering interferometry (BSI), measures small refractive index changes that can accurately quantitate binding events to picomolar concentrations in either a surface-immobilized format or a free solution (Kussrow, A., et al. (2012) Anal Chem. 84, 779-792). While a variety of membrane environments in buffer systems have been used to study ligand-receptor binding affinities (Baksh, M. M., et al. (2011) Nat Biotechnol. 29, 357-360), systems of higher complexity (i.e., physiological matrixes) that would allow for establishment of a meaningful IVIVC have thus far remained elusive.

Accordingly, there is a need in the art for methods, systems, and apparatuses that can provide refractive index related measurements in physiological matrixes.

### SUMMARY

As embodied and broadly described herein, the invention, in one aspect, relates to a method of detecting a binding interaction, the method comprising the steps of: a) preparing a sample comprising uncultured tissue homogenate; b) providing an apparatus adapted for performing light scattering interferometry, the apparatus comprising: i) a fluidic device; ii) a channel formed in the fluidic device capable of receiving the sample and an analyte; iii) a light source for generating a light beam; iv) a photodetector for receiving scattered light and generating intensity signals; and v) at least one signal analyzer capable of receiving the intensity signals and determining therefrom the binding interaction between the sample and the analyte; c) introducing the sample and the analyte into the channel; and d) interrogating the sample using light scattering interferometry.

In one aspect, the invention relates to a method of detecting a binding interaction, the method comprising the steps of: a) preparing a sample comprising uncultured tissue homogenate; b) providing a fluidic device having a channel formed therein for reception of the sample and the analyte; c) introducing the sample and the analyte into the channel; d) directing a light beam from a light source onto the fluidic device such that the light beam is incident on at least a portion of the sample to generate scattered light through reflective and refractive interaction of the light beam with a fluidic device/channel interface, and the sample, wherein the scattered light comprising interference fringe patterns including a plurality of spaced light bands whose positions shift in response to changes in the refractive index of the sample; e) detecting positional shifts in the light bands; and f) determining the binding interaction between the sample and the analyte from the positional shifts of the light bands in the interference fringe patterns.

In one aspect, the invention relates to a method of detecting a binding interaction, the method comprising the steps of: a) preparing a first sample comprising at least one membrane vesicle and a matrix at a first concentration, wherein the matrix is selected from buffer, serum, and/or tissue homogenate; b) preparing a second sample comprising at least one membrane vesicle and a matrix at a second concentration, wherein the matrix is selected from buffer, serum, and/or tissue homogenate and wherein the matrix of the second sample is different than the matrix of the first sample; c) providing an apparatus adapted for performing light scattering interferometry, the apparatus comprising: i) a fluidic device; ii) a channel formed in the fluidic device capable of receiving the first and/or second sample and an analyte; iii) a light source for generating a light beam; iv) a photodetector for receiving scattered light and generating intensity signals; and v) at least one signal analyzer capable of receiving the intensity signals and determining therefrom the binding interaction between the first and/or second sample and the analyte; d) introducing the first and/or second sample and the analyte into the channel; and e) interrogating the first and/or second sample using light scattering interferometry.

In one aspect, the invention relates to a method of detecting a binding interaction, the method comprising the steps of: a) preparing a first sample comprising at least one membrane vesicle and a matrix at a first concentration, wherein the matrix is selected from buffer, serum, and/or tissue homogenate; b) preparing a second sample comprising at least one membrane vesicle and a matrix at a second concentration, wherein the matrix is selected from buffer, serum, and/or tissue homogenate, and wherein the matrix of the second sample is the same as the matrix of the first sample; c) providing an apparatus adapted for performing light scattering interferometry, the apparatus comprising: i) a fluidic device; ii) a channel formed in the fluidic device capable of receiving the first and/or second sample and an analyte; iii) a light source for generating a light beam; iv) a photodetector for receiving scattered light and generating intensity signals; and v) at least one signal analyzer capable of receiving the intensity signals and determining therefrom the binding interaction between the first and/or second sample and the analyte; d) introducing the first and/or second sample and the analyte into the channel; and e) interrogating the first and/or second sample using light scattering interferometry.

In one aspect, the invention relates to a method of predicting the in vivo binding affinity of an analyte, the method comprising the steps of: a) preparing a sample comprising uncultured tissue homogenate; b) providing a fluidic device having a channel formed therein for reception of the sample and the analyte; c) introducing the sample and the analyte into the channel; d) directing a light beam from a light source onto the fluidic device such that the light beam is incident on at least a portion of the sample to generate scattered light through reflective and refractive interaction of the light beam with a fluidic device/channel interface, and the sample, wherein the scattered light comprising interference fringe patterns including a plurality of spaced light bands whose positions shift in response to changes in the refractive index of the sample; e) detecting positional shifts in the light bands; f) determining the K_{D} of the sample and the analyte using the positional shifts in the light bands; and g) predicting the in vivo behavior using the binding affinity.

It will be apparent to those skilled in the art that various devices may be used to carry out the systems, methods, apparatuses, or computer program products of the present invention, including cell phones, personal digital assistants, wireless communication devices, personal computers, or dedicated hardware devices designed specifically to carry out aspects of the present invention. While aspects of the present invention may be described and claimed in a particular statutory class, such as the system statutory class, this is for convenience only and one of skill in the art will understand that each aspect of the present invention can be described and claimed in any statutory class, including systems, apparatuses, methods, and computer program products.

Unless otherwise expressly stated, it is in no way intended that any method or aspect set forth herein be construed as requiring that its steps be performed in a specific order. Accordingly, where a method, system, or computer program product claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE FIGURES

The accompanying figures, which are incorporated in and constitute a part of this specification, illustrate several aspects and together with the description serve to explain the principles of the invention.

FIG. 1 shows a schematic representation of the in vivo status and in vitro components.

FIG. 2 shows representative data pertaining to MAdCAM Ab binding to recombinant MAdCAM in buffer.

FIG. 3 shows the experimental set up for measuring the apparent affinity of anti-MAdCAM MAb to endogenous serum MAdCAM.

FIG. 4 shows representative data pertaining to MAdCAM Ab binding to endogenous serum MAdCAM in 25% serum using MAdCAM Ab with 25% serum stripped of MAdCAM as the reference.

FIG. 5 shows representative data pertaining to MAdCAM Ab binding to endogenous serum MAdCAM in 10% serum.

FIG. 6 shows representative data pertaining to MAdCAM Ab binding to endogenous serum MAdCAM in 25% serum using IL-6 Ab with 25% serum as the reference.

FIG. 7 shows representative data pertaining to MAdCAM Ab binding to endogenous serum MAdCAM in 35% serum.

FIG. 8 shows representative data pertaining to MAdCAM Ab binding to endogenous serum MAdCAM in increasing concentrations of serum.

FIG. 9 shows representative data pertaining to the relationship between serum concentration and MAdCAM Ab affinity.

FIG. 10 shows the cell-based binding experiment design.

FIG. 11 shows representative data pertaining to MAdCAM Ab binding to CHO-MAdCAM cell vesicles in buffer.

FIG. 12 shows representative data pertaining to MAdCAM Ab binding to CHO-MAdCAM cell vesicles in 25% serum.

FIG. 13 shows representative data pertaining to MAdCAM Ab binding to CHO-MAdCAM cell vesicles in 25% tissue homogenate.

FIG. 14 shows the experimental design for measuring the affinity of anti-MAdCAM MAb to both membrane-bound and soluble endogenous MAdCAM.

FIG. 15 shows the tissue-based binding experiment design.

FIG. 16 shows representative data pertaining to MAdCAM Ab binding to human colon tissue vesicles in buffer.

FIG. 17 shows representative data pertaining to MAdCAM Ab binding to human colon tissue vesicles in 25% serum.

FIG. 18 shows representative data pertaining to MAdCAM Ab binding to human colon tissue vesicles in 25% tissue homogenate.

FIG. 19 shows representative data pertaining to MAdCAM Ab binding to human colon tissue vesicles in varying biological matrixes.

FIG. 20 shows representative data summarizing the “true” K_{D}, apparent K_{D}, and integrated K_{D} measured over a range of concentrations and biological matrixes using BSI.

FIG. 21 shows representative data summarizing the BSI measured (red dots), Biacore (black dot), clinically derived (brown dot), and extrapolated (yellow dot) binding affinities.

FIG. 22 shows a cartoon representation pertaining to the apparent K_{D} measured in serum and the integrated Kₐ measured in tissue.

FIG. 23 shows a plot of Target B Serum Binding.

FIG. 24 shows a further plot of Target B Serum Binding.

FIG. 25 shows a plot of Target B Tissue Binding.

FIG. 26 shows a plot of PBMC Vesicle Binding.

FIG. 27 shows a plot of PBMC Whole Cell Binding.

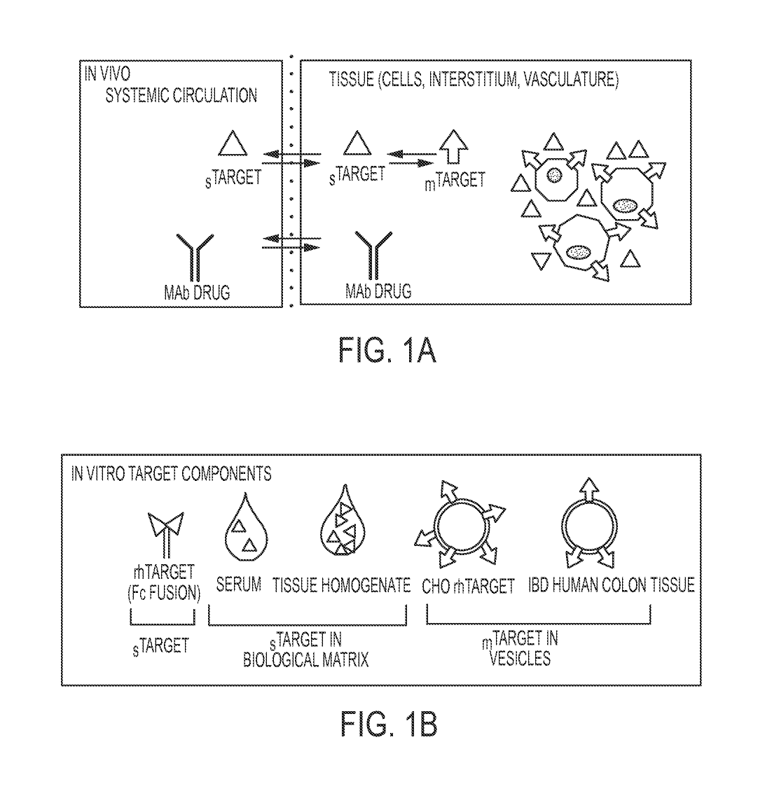

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which can require independent confirmation.

### A. DEFINITIONS

As used in the specification and the appended claims, the singular forms “a,” “an” and “the” include plural referents unless the context clearly dictates otherwise. Thus, for example, reference to “a substrate,” “a polymer,” or “a sample” includes mixtures of two or more such substrates, polymers, or samples, and the like.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the term weight percent (wt %) of a component, unless specifically stated to the contrary, is based on the total weight of the formulation or composition in which the component is included.

As used herein, the terms “optional” or “optionally” means that the subsequently described event or circumstance may or may not occur, and that the description includes instances where the event or circumstance occurs and instances where it does not.

As used herein, the abbreviation “mAb” refers to a monoclonal antibody.

As used herein, the abbreviation “Ab” refers to an antibody.

As used herein, the term “tissue homogenate” refers to an uncultured ex vivo tissue sample comprising whole cells that have been ruptured, allowing release of the intracellular components into the surrounding environment, and further blended into a relatively uniform mass. For example, tissue may be ground with a mortar and pestle. As a further example, tissue may be run through a blender. It is also understood that the tissue homogenate may be further mixed (i.e., centrifuged) to allow for isolation of any remaining whole cells and/or one or more cellular components.

By the term “uncultured tissue,” as used herein, is meant that the tissue sample is not grown separate from the organism from which it is obtained. That is, the sample is not grown or passaged in in vitro culture such that the cells can grow and/or divide before the sample is analyzed. In an uncultured tissue sample, cells that are capable of growing and dividing under tissue culture conditions cannot overgrow the sample such that such cells would be over represented in the sample. Thus, the uncultured tissue sample would be understood to comprise the various components present in the relative proportions as were present in the sample before it was removed from the organism.

As used herein, the term “interstitial environment” refers to the fluid, proteins, solutes, and the extracellular matrix (ECM) that comprise the cellular microenvironment in tissues. Specifically, the interstitial environment can comprise the connective and supporting tissues of the body that are localized outside the blood and lymphatic vessels and parenchymal cells. The interstitial environment can comprise two phases: the interstitial fluid (IF), consisting of interstitial water and its solutes, and the structural molecules of the interstitial or the ECM.

As used herein, the term “chemical event” refers to a change in a physical or chemical property of an analyte in a sample that can be detected by the disclosed systems and methods. For example, a change in refractive index (RI), solute concentration and/or temperature can be a chemical event. As a further example, a biochemical binding or association (e.g., DNA hybridization) between two chemical or biological species can be a chemical event. As a further example, a disassociation of a complex or molecule can also be detected as an RI change. As a further example, a change in temperature, concentration, and association/dissociation can be observed as a function of time. As a further example, bioassays can be performed and can be used to observe a chemical event.

As used herein, the term “drug candidate” refers to a small molecule, an antibody, an antibody fragment, a therapeutic protein, or a therapeutic peptide which can potentially be used as a drug against a disease or condition. The pharmacological activities of the compound may be unknown.

Disclosed are the components to be used to prepare the compositions of the invention as well as the compositions themselves to be used within the methods disclosed herein. These and other materials are disclosed herein, and it is understood that when combinations, subsets, interactions, groups, etc., of these materials are disclosed that while specific reference of each various individual and collective combinations and permutation of these compounds may not be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed.

This concept applies to all aspects of this application including, but not limited to, steps in methods of making and using the compositions of the invention. Thus, if there are a variety of additional steps that can be performed it is understood that each of these additional steps can be performed with any specific aspect or combination of aspects of the methods of the invention.

It is understood that the compositions disclosed herein have certain functions. Disclosed herein are certain structural requirements for performing the disclosed functions and it is understood that there are a variety of structures that can perform the same function that are related to the disclosed structures, and that these structures will typically achieve the same result.

### B. LIGHT SCATTERING INTERFEROMETRY

Rapid monitoring and detection of ultra small volume samples is in great demand. One analytical approach, back-scattering interferometry (BSI), derives from the observation that coherent light impinging on a cylindrically shaped capillary produces a highly modulated interference pattern. Typically, BSI analyzes reflections from a capillary tube filled with a liquid of which one wants to measure the refractive index. The technique has been shown capable of measuring changes in refractive index of liquids on the order of 10⁻⁹. The BSI technique is a simple and universal method of detecting refractive index changes in small volumes of liquid and can be applied to monitor changes in concentrations of solutes, flow rates and temperature, all conducted in nanoliter volumes.

The BSI technique is based on interference of laser light after it is reflected from different regions in a capillary or like sample container. Suitable methods and apparatus are described in U.S. Pat. No. 5,325,170 and WO-A-01/14858, which are hereby incorporated by reference for the purpose of describing methods and apparatus for performing BSI. The reflected or back scattered light is viewed across a range of angles with respect to the laser light path. The reflections generate an interference pattern that moves in relation to such angles upon changing refractive index of the sample. The small angle interference pattern traditionally considered has a repetition frequency in the refractive index space that limits the ability to measure refractive index to refractive index changes causing one such repetition. In one aspect, such refractive index changes are typically on the order of three decades. In another aspect, such changes are on the order of many decades. In another aspect, the fringes can move over many decades up to, for example, the point where the refractive index of the fluid and the channel are matched.

BSI methods direct a coherent light beam along a light path to impinge on a first light transmissive material and pass there through, to pass through a sample which is to be the subject of the measurement, and to impinge on a further light transmissive material, the sample being located between the first and further materials, detecting reflected light over a range of angles with respect to the light path, the reflected light including reflections from interfaces between different substances including interfaces between the first material and the sample and between the sample and the further material which interfere to produce an interference pattern comprising alternating lighter and darker fringes spatially separated according to their angular position with respect to the light path, and conducting an analysis of the interference pattern to determine there from the refractive index, wherein the analysis comprises observation of a parameter of the interference pattern which is quantitatively related to sample refractive index dependent variations in the intensity of reflections of light which has passed through the sample.

The analysis comprises one or both of: (a) the observation of the angle with respect to the light path at which there is an abrupt change in the intensity of the lighter fringes, or (b) the observation of the position of these fringes of a low frequency component of the variation of intensity between the lighter and darker fringes. The first of these (a), relies upon the dependency of the angle at which total internal reflection occurs at an interface between the sample and the further material on the refractive index of the sample. The second (b), relies upon the dependency of the intensity of reflections from that interface on the refractive index as given by the Fresnel coefficients. The rectangular chips also have a single competent from diffraction at the corners.

The first material and the further material are usually composed of the same substance and may be opposite side walls of a container within which the sample is held or conducted. For instance, the sample may be contained in, e.g. flowed through, a capillary dimensioned flow channel such as a capillary tube. The side wall of the capillary tube nearer the light source is then the “first material” and the opposite side wall is the “further material.” The cross-sectional depth of the channel is limited only by the coherence length of the light and its breadth is limited only by the width of the light beam. Preferably, the depth of the channel is from 1 to 10 um, but it may be from 1 to 20 um or up to 50 um or more, e.g. up to 1 mm or more. However, sizes of up to 5 mm or 10 mm or more are possible. Suitably, the breadth of the channel is from 0.5 to 2 times its depth, e.g., equal to its depth.

Typically, at least one the interfaces involving the sample at which light is reflected is curved in a plane containing the light path, the curved interface being convex in the direction facing the incoming light if it is the interface between the first material and the sample and being concave in the direction facing the incoming light if it is the interface between the sample and the further material. The sample is typically a liquid, and can be flowing or stationary. However, the sample can also be a solid or a gas in various aspects of the present invention. The first and/or further materials will normally be solid but in principle can be liquid, e.g., can be formed by a sheathing flow of guidance liquid(s) in a microfluidic device, with the sample being a sheathed flow of liquid between such guidance flows. The sample may also be contained in a flow channel of appropriate dimensions in a fluidic device, such as a microfluidic chip. The method may therefore be employed to obtain a read out of the result of a reaction conducted on a “lab on a chip” type of device.

In contrast to conventional BSI techniques, the present invention provides systems, apparatuses, and methods for the analysis of membrane associated samples, solvents, and systems. In one aspect, the ability to analyze such systems can provide information on chemical and biological interactions previously only attainable by either destructive or complicated, time consuming methods.

### C. APPARATUS FOR PERFORMING LIGHT SCATTERING INTERFEROMETRY

In one aspect, the invention relates to an apparatus adapted for performing light scattering interferometry. Conventional back-scattering interferometry utilizes interference fringes generated by backscattered light to detect refractive index changes in a sample. The backscatter detection technique is generally disclosed in U.S. Pat. No. 5,325,170 to Bornhop, and U.S. Patent Publication No. US2009/0103091 to Bornhop, both of which are hereby incorporated by reference.

In various aspects, the apparatus for performing light scattering interferometry and methods thereof are capable of measuring multiple signals, for example, along a length of a capillary channel, simultaneously or substantially simultaneously. Without wishing to be bound by theory, in various further aspects, the refractive index changes that can be measured by the apparatus and methods of the present disclosure can arise from molecular dipole alterations associated with conformational changes of sample-analyte interaction, as well as density fluctuations.

The apparatus has numerous applications, including the observation and quantification of membrane-associated protein binding events, molecular interactions, molecular concentrations, ligand-metal interactions, electrochemical reactions, ultra micro calorimetry, flow rate sensing, and temperature sensing.

In various aspects, the apparatus and methods described herein can be useful as a bench-top molecular interaction photometer. In a further aspect, the apparatus and methods described herein can be useful for performing bench-top or on-site analysis.

1. Fluidic Device

In one aspect, the apparatus adapted for performing light scattering interferometry comprises a fluidic device. In a further aspect, the fluidic device is a microfluidic device. In a still further aspect, the fluidic device is a microchip.

In various aspects, the fluidic device and channel together comprise a capillary tube. In a further aspect, the fluidic device comprises a silica substrate and an etched channel formed in the device for reception of the sample and/or analyte, the channel having a cross-sectional shape. In a still further aspect, the cross sectional shape of a channel is semi-circular. In yet a further aspect, the cross sectional shape of a channel is square, rectangular, or elliptical. In an even further aspect, the cross sectional shape of a channel can comprise any shape suitable for use in a BSI technique. In a still further aspect, a fluidic device can comprise one or multiple channels of the same or varying dimensions.

In various aspects, the material of composition of the fluidic device has a different index of refraction than that of the sample to be analyzed. In a further aspect, as refractive index can vary significantly with temperature, the fluidic device can optionally be mounted and/or connected to a temperature control device. In a still further aspect, the fluidic device can be tilted, for example, about 7°, such that scattered light from channel can be directed to a detector.

2. Channel

In one aspect, the apparatus adapted for performing light scattering interferometry comprises a channel formed in the fluidic device capable of receiving the sample and an analyte. The channel of the present invention can, in various aspects, be formed from the fluidic device, such as a piece of silica or other suitable optically transmissive material. In various aspects, the channel has a generally semi-circular cross-sectional shape. A unique multi-pass optical configuration is inherently created by the channel characteristics, and is based on the interaction of the unfocused laser beam and the curved surface of the channel that allows interferometric measurements in small volumes at high sensitivity. Alternatively, the channel can have a substantially circular or generally rectangular cross-sectional shape.

In various aspects, the channel can have a radius of from about 5 to about 250 micrometers, for example, about 5, 10, 20, 30, 40, 50, 75, 100, 150, 200, or 250 micrometers. In a still further aspect, the channel can have a radius of up to about 1 millimeter or larger, such as, for example, 0.5 millimeters, 0.75 millimeters, 1 millimeter, 1.25 millimeters, 1.5 millimeters, 1.75 millimeters, 2 millimeters, or more.

In various aspects, the channel can hold and/or transport the same or varying samples, and a mixing zone. The design of a mixing zone can allow at least initial mixing of, for example, one or more binding pair species. In a further aspect, the at least initially mixed sample can then optionally be subjected to a stop-flow analysis, provided that the reaction and/or interaction between the binding pair species continues or is not complete at the time of analysis. The specific design of a fluidic channel, mixing zone, and the conditions of mixing can vary, depending on such factors as, for example, the concentration, response, and volume of a sample and/or species, and one of skill in the art, in possession of this disclosure, could readily determine an appropriate design.

In various aspects, a channel comprises a single zone along its length for analysis. In a further aspect, a channel can be divided into multiple discrete zones along the length of the channel, such as, for example, 2, 3, 4, 5, 6, 7, 8, 9, 10, or more zones. If a channel is divided into zones, any individual zone can have dimensions, such as, for example, length, the same as or different from any other zones along the same channel. In a still further aspect, at least two zones have the same length. In yet a further aspect, all of the zones along the channel have the same or substantially the same length. In an even further aspect, each zone can have a length along the channel of from about 1 to about 1,000 micrometers, for example, about 1, 2, 3, 5, 8, 10, 20, 40, 80, 100, 200, 400, 800, or 1,000 micrometers. In a still further aspect, each zone can have a length of less than about 1 micrometer or greater than about 1,000 micrometer, and the present disclosure is not intended to be limited to any particular zone dimension. In yet a further aspect, at least one zone can be used as a reference and/or experimental control. In an even further aspect, each measurement zone can be positioned adjacent to a reference zone, such that the channel comprises alternating measurement and reference zones. It should be noted that the zones along a channel do not need to be specifically marked or delineated, only that the system be capable of addressing and detecting scattered light from each zone.

In various aspects, any one or more zones in a channel can be separated from any other zones by a junction, such as, for example, a union, coupling, tee, injection port, mixing port, or a combination thereof. For example, one or more zones in the flow path of a sample can be positioned upstream of an injection port where, for example, an analyte can be introduced. In such an aspect, one or more zones can also be positioned downstream of the injection port.

In various aspects, a channel can be divided into two, three, or more regions, wherein each region is separated from other regions by a separator. In a further aspect, a separator can prevent a fluid in one region of a channel from contacting and/or mixing with a fluid from another region of the channel. In a still further aspect, any combination of regions or all of the regions can be positioned such that they will be impinged with at least a portion of the light beam. In such an aspect, multiple regions of a single channel can be used to conduct multiple analyses of the same of different type in a single instrumental setup. In yet a further aspect, a channel has two regions, wherein a separator is positioned in the channel between the two regions, and wherein each of the regions are at least partially in an area of the channel where the light beam is incident.

In various aspects, if multiple regions are present, each region can have an input and an output port. In a further aspect, the input and/or output ports can be configured so as not to interfere with the generation of scattered light, such as, for example, back-scattered light, and the resulting measurements. It should be noted that other geometric designs and configurations can be utilized, and the present invention is not intended to be limited to the specific exemplary configurations disclosed herein. Thus, various further aspects, a single channel can allow for analysis of multiple samples simultaneously in the same physical environment.

In various aspects, a separator, if present, comprises a material that does not adversely affect detection in each of the separated regions, such as, for example, by creating spurious light reflections and refractions. In a further aspect, a separator is optically transparent. In a still further aspect, a separator does not reflect light from the light source. In such an aspect, a separator can have a flat black, non-reflective surface. In yet a further aspect, the separator can have the same or substantially the same index of refraction as the channel. In an even further aspect, a separator can be thin, such as, for example, less than about 2 μm, less than about 1 μm, less than about 0.75 μm.

Any one or more individual zones along the channel, or any portion of the channel, can optionally comprise a marker compound positioned within the path of the channel. In various aspects, a marker compound can be positioned on the interior surface of a capillary such that a sample, when introduced into the channel, can contact and/or interact with the marker compound.

A marker compound, if present, can comprise any compound capable of reacting or interacting with a sample or an analyte species of interest. In various aspects, a marker compound can comprise a chromophore. In a further aspect, a marker compound can comprise a ligand that can interact with a species of interest to provide a detectable change in refractive index.

As the light beam impinges one or more discrete regions of a channel, the resulting interference fringe patterns can move with a change in refractive index. The ability to analyze multiple discrete zones simultaneously can provide high spatial resolution and can provide measurement techniques with an integrated reference.

3. Photodetector

In one aspect, the apparatus adapted for performing light scattering interferometry comprises a photodetector for receiving scattered light and generating intensity signals. A photodetector detects the scattered light and converts it into intensity signals that vary as the positions of the light bands in the elongated fringe patterns shift, and can thus be employed to determine the refractive index (RI), or an RI related characteristic property, of the sample. The photodetector can, in various aspects, comprise any suitable image sensing device, such as, for example, a bi-cell sensor, a linear or area array CCD or CMOS camera and laser beam analyzer assembly, a photodetector assembly, an avalanche photodiode, or other suitable photodetection device. In a further aspect, the photodetector is an array photodetector capable of detecting multiple interference fringe patterns. In a still further aspect, a photodetector can comprise multiple individual detectors to detect interference fringe patterns produced by the interaction of the light beam with the sample, channel wall, and optional marker compounds. In yet a further aspect, the scattered light incident upon the photodetector comprises interference fringe patterns. In an even further aspect, the scattered light incident upon the photodetector comprises elongated interference fringe patterns that correspond to the discrete zones along the length of the channel. The specific position of the detector can vary depending upon the arrangement of other elements. In a still further aspect, the photodetector can be positioned at an approximately 45° angle to the channel.

4. Signal Analyzer

In one aspect, the apparatus adapted for performing light scattering interferometry comprises at least one signal analyzer capable of receiving the intensity signals and determining therefrom the binding interaction between the sample and the analyte. The intensity signals from the photodetector can then be directed to a signal analyzer for fringe pattern analysis and determination of the RI or RI related characteristic property of the sample and/or reference in each zone of the channel. The signal analyzer can be a computer or a dedicated electrical circuit. In various aspects, the signal analyzer includes the programming or circuitry necessary to determine from the intensity signals, the RI or other characteristic property of the sample in each discrete zone of interest. In a further aspect, the signal analyzer is capable of detecting positional shifts in interference fringe patterns and correlating those positional shifts with a change in the refractive index of at least a portion of the sample. In a still further aspect, the signal analyzer is capable of detecting positional shifts in interference fringe patterns and correlating those positional shifts with a change in the refractive index occurring in a portion of the channel. In yet a further aspect, the signal analyzer is capable of comparing data received from a detector and determining the refractive index and/or a characteristic property of the sample in any zone or portion of the channel.

In various aspects, the signal analyzer is capable of interpreting an intensity signal received from a detector and determining one or more characteristic properties of the sample. In a further aspect, the signal analyzer can utilize a mathematical algorithm to interpret positional shifts in the interference fringe patterns incident on a detector. In yet a further aspect, known mathematical algorithms and/or signal analysis software, such as, for example, deconvolution algorithms, can be utilized to interpret positional shifts occurring from a multiplexed scattering interferometric analysis.

The detector can be employed for any application that requires interferometric measurements; however, the detector can be particularly useful for making universal solute quantification, temperature and flow rate measurements. In these applications, the detector provides ultra-high sensitivity due to the multi-pass optical configuration of the channel. In the temperature measuring aspect, a signal analyzer receives the signals generated by the photodetector and analyzes them using the principle that the refractive index of the sample varies proportionally to its temperature. In this manner, the signal analyzer can calculate temperature changes in the sample from positional shifts in the detected interference fringe patterns. In various aspects, the ability to detect interference fringe patterns from interactions occurring along a channel can provide real-time reference and/or comparative measurements without the problem of changing conditions between measurements. In a further aspect, a signal analyzer, such as a computer or an electrical circuit, can thus be employed to analyze the photodetector signals, and determine the characteristic property of the sample.

In the flow measuring aspect, the same principle is also employed by the signal analyzer to identify a point in time at which perturbation is detected in a flow stream in the channel. In the case of a thermal perturbation, a flow stream whose flow rate is to be determined, is locally heated at a point that is known distance along the channel from the detection zone. The signal analyzer for this aspect includes a timing means or circuit that notes the time at which the flow stream heating occurs. Then, the signal analyzer determines from the positional shifts of the light bands in the interference fringe patterns, the time at which thermal perturbation in the flow stream arrives at the detection zone. The signal analyzer can then determine the flow rate from the time interval and distance values. Other perturbations to the flow stream, include, but are not limited to, introduction into the stream of small physical objects, such as glass microbeads or nanoparticles. Heating of gold particles in response to a chemical reaction or by the change in absorption of light due to surface-bound solutes or the capture of targets contained within the solution can be used to enhance the temperature induced RI perturbation and thus to interrogate the composition of the sample. In various aspects, measurements at multiple zones along the channel can be used to determine temperature gradients or rate of temperature change of a sample within the channel.

In various aspects, the systems and methods of the present invention can be used to obtain multiple measurements simultaneously or substantially simultaneously from discrete zones along the length of a channel. In such an aspect, each zone can provide a unique measurement and/or reference. For example, a series of reactive species can be used as marker compounds, positioned in zones along the channel, each separated by a reference zone. In a further aspect, temporal detection can be used to measure changes in a sample over time as the sample flows through the channel, for example, with a flow injection analysis system.

In various aspects, two or more samples, blanks, and/or references can be positioned in the channel such that they are separated by, for example, an air bubble. In a further aspect, each of a plurality of samples and/or reference species can exhibit a polarity and/or refractive index the same as or different from any other samples and/or reference species. In a still further aspect, a pipette can be used to place a portion of a reference compound into the channel. Upon removal of the pipette, an air bubble can be inserted between the portion of the reference compound in the channel and a portion of a sample compound, thereby separating the reference and sample compounds and allowing for detection of each in a flowing stream within the channel. In yet a further aspect, each sample and/or reference compound can be separated by a substance other than air, such as, for example, water, oil, or other solvent having a polarity such that the sample and/or reference compounds are not miscible therewith.

5. Light Source

In one aspect, the apparatus adapted for performing light scattering interferometry comprises a light source for generating a light beam. In a further aspect, the light source generates an easy to align optical beam that is incident on the etched channel for generating scattered light. In a still further aspect, the light source generates an optical beam that is collimated, such as, for example, the light emitted from a HeNe laser. In yet a further aspect, the light source generates an optical beam that is not well collimated and disperses in, for example, a Gaussian profile, such as that generated by a diode laser. In an even further aspect, at least a portion of the light beam is incident on the channel such that the intensity of the light on any one or more zones is the same or substantially the same. In a still further aspect, the portion of the light beam incident on the channel can have a non-Gaussian profile, such as, for example, a plateau (e.g., top-hat). The portion of the light beam in the wings of the Gaussian intensity profile can be incident upon other portions of the channel or can be directed elsewhere. In yet a further aspect, variations in light intensity across the channel can result in measurement errors. In an even further aspect, if portions of a light beam having varying intensity are incident upon multiple zones or portions of a channel, a calibration can be performed wherein the expected intensity of light, resulting interaction, and scattering is determined for correlation of future measurements.

The light source can comprise any suitable equipment and/or means for generating light, provided that the frequency and intensity of the generated light are sufficient to interact with a sample and/or a marker compound and provide elongated fringe patterns as described herein. Light sources, such as HeNe lasers and diode lasers, are commercially available and one of skill in the art could readily select an appropriate light source for use with the systems and methods of the present invention. In various aspects, a light source can comprise a single laser. In a further aspect, a light source can comprise two or more lasers, each generating a beam that can impinge one or more zones of a channel. In a still further aspect, if two or more lasers are present, any individual laser can be the same as or different from any other laser. For example, two individual lasers can be utilized, each producing a light beam having different properties, such as, for example, wavelength, such that different interactions can be determined in each zone along a channel.

As with any interferometric technique for micro-chemical analysis, it can be advantageous, in various aspects, for the light source to have monochromaticity and a high photon flux. If warranted, the intensity of a light source, such as a laser, can be reduced using neutral density filters.

The systems and methods of the present invention can optionally comprise an optical element that can focus, disperse, split, and/or raster a light beam. In various aspects, an optical element, if present, can at least partially focus a light beam onto a portion of the channel. In a further aspect, such an optical element can facilitate contact of the light beam with one or more zones along a channel. In a still further aspect, a light source, such as a diode laser, generates a light beam having a Gaussian profile, and an optical element is not necessary or present. In yet a further aspect, a light source, such as a diode laser, can be used together with an optical focusing element. In an even further aspect, a light source, such as a HeNe laser, generates a collimated light beam and an optical element can be present to spread the light beam, for example, to a degree greater than any naturally occurring dispersion, and facilitate contact of the light beam with at least two zones along the channel. In another aspect, an optical element can be used to spread or disperse a light beam in one direction, such that the resulting beam has a larger dimension in a first direction than in a perpendicular direction. Such a light beam configuration can allow for multiple measurements or sample and reference measurements to be made simultaneously or substantially simultaneously within the same channel.

In various aspects, an optical element, if present, can comprise a dispersing element, such as a cylindrical lens, capable of dispersing the light beam in at least one direction; an anamorphic lens; a beam splitting element capable of splitting a well collimated light beam into two or more individual beams, each of which can be incident upon a separate zone on the same channel; a rastering element capable of rastering a light beam across one or more zones of a channel; or a combination thereof.

In various aspects, one or more additional optical components can be present, such as, for example, a mirror, a neutral density filter, or a combination thereof, so as to direct the light beam and/or the scattered light in a desired direction or to adjust one or more properties of a light beam.

In a further aspect, the light source comprises a HeNe laser or a diode laser. In a still further aspect, the laser emits light at from about 10⁻⁵ mW to about 10 mW. In yet a further aspect, the laser emits light at from about 10⁻⁴ mW to about 10 mW. In an even further aspect, the laser emits light at from about 0.01 mW to about 10 mW. In a still further aspect, the laser emits light at from about 0.1 mW to about 10 mW. In yet a further aspect, the laser emits light at from about 1 mW to about 10 mW. In an even further aspect, the laser emits light at from about 10⁻⁵ mW to about 1 mW. In a still further aspect, the laser emits light at from about 10⁻⁵ mW to about 0.1 mW. In yet a further aspect, the laser emits light at from about 10⁻⁵ mW to about 0.01 mW. In an even further aspect, the laser emits light at from about 10⁻⁵ mW to about 10⁻⁴ mW.

### D. PREPARATION OF TISSUE SAMPLES

In one aspect, the invention relates to the preparation of a sample comprising uncultured tissue homogenate. Without wishing to be bound by theory, samples can be prepared using any conventional methods or combinations of methods known to those of skill in the art (see, i.e., U.S. patent application Ser. No. 12/799,689; WO 2012/060882 A2; U.S. patent application Ser. No. 13/409,557).

In one aspect, the invention relates to the preparation of a sample comprising uncultured tissue homogenate. The term “tissue homogenate”, as used herein, refers to an ex vivo tissue sample obtained from a subject comprising whole cells that have been ruptured, allowing release of the intracellular components into the surrounding environment, and further ground into a relatively uniform mass. For example, tissue may be ground with a mortar and pestle. As a further example, tissue may be run through a blender. It is also understood that the tissue homogenate may be further mixed (i.e., centrifuged) to allow for isolation of any remaining whole cells and/or one or more cellular components.

In a further aspect, the tissue homogenate comprises at least one membrane vesicle and/or an interstitial environment. In a still further aspect, the tissue homogenate comprises at least one membrane vesicle. In yet a further aspect, the tissue homogenate comprises an interstitial environment. In an even further aspect, the tissue homogenate comprises at least one membrane vesicle and an interstitial environment.

In a further aspect, the tissue homogenate comprises at least one of a protein, small molecule, nucleic acid, polypeptide, carbohydrate, lipid, glycoprotein, lipoprotein, DNA, RNA, DNA-protein construct, or RNA-protein construct.

In a further aspect, the tissue homogenate comprises at least one endogenous protein. In a still further aspect, the endogenous protein is soluble and/or membrane bound. In yet a further aspect, the endogenous protein is soluble and membrane bound. In an even further aspect, the endogenous protein is membrane bound.

In a further aspect, the endogenous protein is selected from a G-protein coupled receptor, an ion-channel receptor, a tyrosine kinase-linked receptor, and a cytokine receptor.

In various aspects, the sample is a fluid. In a further aspect, the sample is a liquid, which can be a substantially pure liquid, a solution, or a mixture. In a still further aspect, the sample further comprises one or more analytes. In yet a further aspect, a sample can be introduced into the channel via an injection port at, for example, one end of the channel.

The methods and techniques described herein can be performed for any system and/or analyte species. In another aspect, the BSI techniques described herein can be performed in an aqueous system, a non-aqueous system, or a mixture of aqueous and non-aqueous components. In another aspect, a solvent and/or sample can comprise a mixture of two or more solvents having the same or different polarities. In another aspect, a solvent mixture can be selected based on, for example, Hansen solubility parameters, so as to be compatible with one or more analytes of interest. In yet another aspect, the composition of a solvent can be adjusted during the course of an analysis so as to provide, for example, a gradient.

1. Subjects

In various aspects, the tissue homogenate can be obtained from a subject. As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. “Subject” includes both living and nonliving animals and includes patients, healthy subjects, and cadavers. A patient refers to a subject afflicted with a disease or disorder. The term “patient” includes human and veterinary subjects. A healthy subject is a subject not yet diagnosed with a disease or disorder. Nonhuman subjects include livestock (e.g., sheep and cows), poultry (e.g., turkeys and chickens), farmed fish, pets (e.g., dogs and cats), and test subjects (e.g., mice, rats, monkeys, dogs, zebrafish, and chicken embryos).

2. Obtaining a Sample

In one aspect, the invention relates to the collection of a sample comprising cellular content. Without wishing to be bound by theory, samples can be collected using any conventional methods or combinations of methods known to those of skill in the art. In a further aspect, the sample can be collected from almost any source, including without limitation, humans, animals, and the environment.

In one aspect, the sample can comprise a tissue sample and/or liquid sample. In a further aspect, the liquid sample can be obtained by invasive techniques, for example and without limitation, by venipuncture in the case of blood or lumbar puncture in the case of cerebrospinal fluid (CSF). In a further aspect, the sample can be a fluid sample, for example a fluid expressed from the body (e.g., colostrum). In another aspect, the liquid sample can be obtained by non-invasive techniques, for example, as with urine, or using rinses of various body parts or cavities, including but not limited to lavages and mouthwashes. In one aspect, the liquid sample can be collected using a rinse or lavage, and refers to the use of a volume of liquid to wash over or through a body part or cavity, resulting in a mixture of liquid and cells from the body part or cavity.

In various aspects, the tissue sample is collected by biopsy, which can, for example, be done by an open or percutaneous technique. In one aspect, the tissue sample can be collected by open biopsy, which is an invasive surgical procedure using a scalpel and involving direct vision of the target area. In a further aspect, the tissue sample can comprise an entire mass (excisional biopsy) or a part of a mass (incisional biopsy). In one aspect, the tissue sample can be collected by disposing a collection device proximate to and/or within a tissue, such as of a body, drawing in at least a portion of the tissue into the collection device, adhering to at least a portion of the tissue to at least a portion of the collection device and separating the sample and collection device from the remainder of the tissue and/or body.

In another aspect, the tissue sample can be collected by percutaneous biopsy, which can, for example, be performed using a needle-like instrument through a relatively small incision, blindly or with the aid of an imaging device. In a further aspect, the percutaneous biopsy is a fine needle aspiration (FNA) biospy, where, for example, individual cells or clusters of cells are collected for preparation and examination. In a still further aspect, the percutaneous biopsy is a core biopsy, where, for example, a core or fragment of tissue is obtained, and which may be done via a frozen section or paraffin section. In one aspect, the tissue sample can include inserting a coring biopsy needle into a tissue or body and positioning the distal end of the coring needle proximate to and/or within a target tissue.

In one aspect, the whole sample collected can be utilized in the present method. In a further aspect, an extracted component of the sample is utilized, for example, in cases where the desired component is cellular or subcellular. In a still further aspect, the tissue sample can comprise connective, muscle, nervous, or epithelial tissue, or a combination thereof.

In a further aspect, the liquid sample can comprise intracellular fluid or extracellular fluid, for example, and without limitation, intravascular fluid (blood plasma), interstitial fluid, lymphatic fluid, and transcellular fluid. In a yet further aspect, the liquid sample can comprise amniotic fluid, aqueous humour, vitreous humour, bile, whole blood, blood serum/plasma, colostrum, cerebrospinal fluid, chyle, chyme endolymph, perilymph, exudates, feces, gastric acid, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit, or a combination thereof.

3. Membrane Vesicles

In various aspects, the tissue homogenate comprises at least one membrane vesicle. Samples comprising membranes from cells for use in any of the disclosed methods can be prepared by methods known in the art. In one aspect, tissue can harvested from a subject. Tissue can be solubilized or suspended in an appropriate buffer, cleaned and isolated, e.g., by centrifugation. The cells are fragmented by homogenation, shearing, other mechanical methods or similar methods. Membrane materials are washed and isolated, e.g., by centrifugation. Then the membranes are re-suspended in an appropriate buffer. Sample protocols for preparing membrane vesicles are provided in the Examples.

In various aspects, the membrane vesicles comprise native membrane vesicles. The native membrane vesicle sample can be prepared from cultured animal cells or cell lines. Any animal cell or cell line can be used in the sample preparation methods described herein. For example, and not intending to be limiting, in one aspect the cells can be adherent cells, such as, for example, Chinese hamster ovary (CHO-K1) cells. In a further aspect, the cells can be suspension cells, such as suspension human T-lymphocytes (SUP-T1). In a still further aspect, CXCR4-positive cells, CXCR4-negative SUP-T1 cells, or a combination thereof can be used in the methods described herein. Additional cell lines that can be used in the methods described herein, include, but are not limited to, 293-T, 3T3, 721, 9L, A2780, A2780ADR, A2780cis, A172, A20, A253, A431, A-549, ALC, B16, B35, BCP-1, BEAS-2B, bEnd.3, BHK-21, BR 293, BxPC3, C3H-10T1/2, C6/36, Cal-27, CEM, CEM-SS, CHO, COR-L23, COR-L23/CPR, COR-L23/5010, COR-L23/R23, COS, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hep-2, Hepa1c1c7, Hep-G2, HL-60, HMEC, HT-29, Huh-7, Jurkat, JY, K562, Ku812, KCL22, KG1, KYO1, LNCap, Ma-Mel 1, 2, 3 . . . 48, MC-38, MCF-7, MCF-10A, MDA-MB-231, MDA-MB-468, MDA-MB-435, MDCK, MDCK II, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN, OPCT, Peer, PNT-1A, PNT 2, RenCa, RIN-5F, RMA/RMAS, Saos-2, Sf-9, SkBr3, T2, T-47D, T84, THP1, TZM-bl, U373, U87, U937, VCaP, Vero, WM39, WT-49, X63, YAC-1, YAR cells, or a combination thereof. The cells can be wild type cells or cells engineered to express specific proteins, including, but not limited to, full length transmembrane B-forms of both the rat and human gamma-aminobutyric acid receptor (GABAB) or zinc finger nuclease. Optionally, when the cultured cells are engineered to express a specific protein, the expression of the protein can be verified by Western immunoblotting using standard techniques known to a person of ordinary skill in the art. In a still further aspect, the cells can be primary cells. Primary cells can be cells cultured directly from a subject. Primary cells can include, but are not limited to, human hepatocytes, primary fibroblasts, or peripheral blood mononuclear cells (PBMCs).

In various aspects, the preparation of native membrane vesicle samples can include obtaining a pre-cultured population of cells. As used herein, a “pre-cultured population of cells” can be a population of cells already grown to the proper concentrations suitable for use in the methods described herein. In a further aspect, the method of preparing native membrane vesicle samples from cultured cells can include the first step of growing, or culturing, the cells. The cultured cells can be adherent or suspension cells, and either type of cell can be cultured in any growth media appropriate for the cell or cell line being cultured. Growth media that can be used in the methods described herein includes, but is not limited to, RPMI 1640, MEM, DMEM, EMEM, F-10, F-12, Medium 199, MCDB131, or L-15. In a still further aspect, the growth media can be supplemented with components that enhance cell growth. Media supplements that can be used in the methods described herein include, but are not limited to, animal serum, such as fetal bovine serum or fetal calf serum, animal digests, such as proteose peptone, buffers, amino acids, vitamins, antibiotics, or antifungal compounds. A person having ordinary skill in the art can readily determine the appropriate type of growth media and media supplements necessary to support the growth of the cell or cell line being cultured. Growth conditions can vary depending on the cell or cell line being cultured; however, generally, adherent cells can be grown at about 37° C. and about 5% ambient CO₂ to about 100% confluence for about three days once the cells are added to a cell culture flask. The cell culture flask can be a 25 cm², a 75 cm², a 150 cm², or a 175 cm²-area flask, or any other size flask used to culture cells or cell lines. Once adherent cells reach about 100% confluence, they can be harvested by removing all growth media from the flask and incubating with an appropriate volume of a cell detachment solution, such as Detachin solution or trypsin solution, for about 5 min at about 37° C. The appropriate volume of cell detachment solution can vary depending on the size of the cell culture flask being used. For example, about 3 mL of cell detachment solution can be used when cells are cultured in a 75 cm²-area flask, whereas about 4 mL cell detachment solution can be used when cells are cultured in a larger flask, such as a 150 cm² or a 175 cm²-area flask. About 50 mL of incubation buffer can then be added to the flask and the contents can be removed and transferred to two 50 mL centrifuge tubes. As used herein, a “centrifuge tube” can be any tapered tube of any size, which can be made of glass or plastic. The capacity of the centrifuge tube can be, but is not limited to, less than 100 μL, 100 μL, 200 μL, 250 μL, 500 μL, 1 mL, 2 mL, 2.5 mL, 5 mL, 10 mL, 15 mL, 25 mL, 50 mL, 100 mL, greater than 100 mL, or any capacity in between. A centrifuge tube can also be a microcentrifuge tube.

In various aspects, suspension cells can be used in the methods described herein. Growth conditions can vary depending on the cell or cell line being cultured; however, generally, suspension cells can be grown at about 37° C. and about 5% ambient CO₂ to an approximate concentration of about 300,000 cells/mL, using growth media appropriate for the cell or cell line being cultured. The cell culture flask can be a 25 cm², a 75 cm², a 150 cm², or a 175 cm²-area flask, or any other size flask used to culture cells or cell lines. Once the adherent cells have been harvested or the suspension cells have reached a concentration of about 300,000 cells/mL, the cell solution can be centrifuged for about 5 min at about 300 g to pellet the cells; however, the time and rate of centrifugation can be adjusted according to the type of cell or cell line sample being prepared. Following centrifugation, the incubation buffer or media can be removed from the centrifuge tubes, the cells can be re-suspended in a buffer solution suitable for cell culture, for example PBS 1×, and the cell/buffer suspension can be re-centrifuged. Cell pellets can be rinsed once, twice, three times, or more than three times in PBS 1×, each time being re-centrifuged, then can be used immediately to prepare native membrane vesicles for analysis using BSI.

In various aspects, following centrifugation of the cultured cells, the cell pellet can be re-suspended in about 20 mL of ice-cold lysis buffer and placed on a rotator for about 45 minutes at about 4° C. Any lysis buffer known in the art can be used in the methods described herein. In a further aspect, the lysis buffer can comprise 2.5 mM NaCl, 1 mM Tris, and 1×EDTA-free broad-spectrum protease inhibitors, and can be at about pH 8.0. The cell pellet can contain about 10⁶ cultured cells. The resulting solution can then be centrifuged at from about 8,000 g to about 10,000 g for about 60 min at about 4° C.; however, the time and rate of centrifugation can be adjusted according to the type of cell pellet sample being prepared. The supernatant can be removed and the pellet can be re-suspended in about 4 mL of ice-cold, buffer, for example, PBS 1×, then transferred to a new container. In a still further aspect, the container can be a 5 mL glass dram vial. The pellet and buffer can then be sonicated to clarity in an ice bath. Any means for sonication can be used in the methods described herein. For example, sonication can be applied using an ultrasonic bath, known as bath sonication, or an ultrasonic probe, known as probe sonication. The resulting solutions can be centrifuged for about 1 hour at about 16,000 g and about 4° C.; however, the time and rate of centrifugation can be adjusted according to the type of cell pellet sample being prepared. The sizes of the native membrane vesicles collected can then be determined by dynamic light scattering. In yet a further aspect, sizes of the native membrane vesicles can be determined using a Wyatt Technologies DynaPro dynamic light scattering apparatus. If not being analyzed by BSI immediately upon sample preparation, the native membrane vesicle samples can be stored at about 4° C. for about two days, and then analyzed using BSI.

In various aspects, the native membrane vesicle sample can be prepared without the use of lysis buffer, wherein, following centrifugation of the cells, the cell pellet can be re-suspended in about 20 mL of ice-cold buffer containing 2×EDTA-free broad spectrum protease inhibitors. The cell pellet can contain about 10⁶ cultured cells. The resulting solution can then be centrifuged at about 40,000 g for about 60 min at about 4° C.; however, the time and rate of centrifugation can be adjusted according to the type of cell pellet sample being prepared. The supernatant can be removed and the pellet can be re-suspended in about 4 mL of ice-cold, buffer, for example, PBS 1×, and then transferred to a new container. In a further aspect, the container can be a 5 mL glass dram vial. The pellet and buffer can then be sonicated to clarity in an ice bath and transferred to a centrifuge tube filter, for example, and not to be limiting, a 220 nm Millipore Ultrafree-MC centrifuge tube filter. Any means for sonication can be used in the methods described herein. For example, and not to be limiting, sonication can be applied using an ultrasonic bath, known as bath sonication, or an ultrasonic probe, known as probe sonication. The resulting solutions can be centrifuged for about 1 h at about 16,000 g and about 4° C.; however, the time and rate of centrifugation can be adjusted according to the type of pellet being used. Native membrane vesicles can be collected by capturing the solution that passes through the centrifuge tube filter, and the sizes of the native membrane vesicles collected can be determined by dynamic light scattering. In one aspect, sizes of the native membrane vesicles can be determined using a Wyatt Technologies DynaPro dynamic light scattering apparatus. If not being analyzed by BSI immediately upon sample preparation, the native membrane vesicle samples can be stored at about 4° C. for about two days, and then analyzed using BSI.

In various aspects, the membrane vesicles comprise synthetic membranes. Small unilamellar vesicles (SUV) can be formed using standard techniques known in the art. For example, a lipid solution in chloroform can be evaporated in a flask, for example, a small round-bottom flask, and then hydrated for about 1 hour at about 4° C. in deionized (18.2 MW-cm) water, 0.5×PBS or 1×PBS at ˜3.3 mg/mL. Lipids that can be used in the methods described herein include, but are not limited to, 1,2-dimyristoleoyl-sn-glycero-3-phosphocholine (DMOPC) and 1,2-dimyristoyl-sn-glycero-3-[phospho-L-serine] (sodium salt) (DMPS). In a further aspect, the deionized water can be Milli-Q deionized (18.2 MW-cm) water. The lipids can be sonicated to clarity in an ice-water bath and transferred to a centrifuge tube filter, for example, a 100 nm Millipore Ultrafree-MC centrifuge tube filter. Any means for disruption, for example, sonication, can be used in the methods described herein. For example, sonication can be applied using an ultrasonic bath, known as bath sonication, or an ultrasonic probe, known as probe sonication. Samples can then be centrifuged for about 2 hours at about 16,000 g and about 4° C.; however, the time and rate of centrifugation can be adjusted according to the type of synthetic membrane vesicle sample being prepared. Synthetic membrane vesicles can be collected by capturing the solution that passes through the centrifuge tube filter, and the sizes of the synthetic membrane vesicles collected can be determined by dynamic light scattering. In a still further aspect, sizes of the synthetic membrane vesicles can be determined using a Wyatt Technologies DynaPro dynamic light scattering apparatus. If not being analyzed using BSI immediately upon sample preparation, the synthetic membrane vesicle samples can be stored at about 4° C. for about one week.

In various aspects, full-length fatty acid amide hydrolase (FAAH), a transmembrane protein important in neurological function and a drug target for pain management and other indications, can be incorporated into synthetic lipid vesicles by mixing FAAH, which can be reconstituted in 1% w/v n-octyl-beta-D-glucopyranoside (n-OG) in 1×PBS, and SUVs to a final concentration of about 100 μg of protein per mL of centrifuged SUV solution. The resulting mixture can then be dialyzed against either 1×PBS, pH 7.4 or 100 mM Tris pH 9.0 to facilitate complete removal of detergent. The size of the resulting proteoliposomes can be measured by dynamic light scattering. In a further aspect, the lipid: protein ratio can be about 3300:1. In a still further aspect, the proteoliposomes can be about 150 nm in diameter. If not being analyzed by BSI immediately upon sample preparation, proteoliposomes can be stored at about 4° C. for about one week, and then analyzed using BSI.

In various aspects, the membrane vesicles comprise one or more native membrane vesicle samples, one or more synthetic membrane vesicle samples, or a combination thereof.

4. Interstitial Environment

In various aspects, the tissue homogenate comprises an interstitial environment. In a further aspect, the tissue homogenate comprises at least one membrane vesicle and an interstitial environment. Without wishing to be bound by theory, the term “interstitial environment” refers to the fluid, proteins, solutes, and the extracellular matrix (ECM) that comprise the cellular microenvironment in tissues. Specifically, the interstitial environment can comprise the connective and supporting tissues of the body that are localized outside the blood and lymphatic vessels and parenchymal cells. It can comprise two phases: the interstitial fluid (IF), consisting of interstitial water and its solutes, and the structural molecules of the interstitial or the ECM.

Examples of interstitial environments may include, but are not limited to, blood plasma, lymph, synovial fluid, cerebrospinal fluid, aqueous and vitreous humor, serous fluid, and fluid secreted by glands, or a mixture thereof. In various aspects, the interstitial environment may comprise sugars, salts, fatty acids, amino acids, coenzymes, hormones, neurotransmitters, as well as waste products from cells.

### E. ANALYTES

In one aspect, the invention relates to methods of detecting a binding interaction between a sample and an analyte. In a further aspect, the sample further comprises the analyte. Such methods are useful in drug discovery in which drug candidates are tested for their ability to bind a component of the sample of interest. In various aspects, the term “drug candidate” refers to a small molecule, an antibody, an antibody fragment, a therapeutic protein, or a therapeutic peptide which can potentially be used as a drug against a disease or condition. The pharmacological activities of the compound can be known, partially known, or unknown.

Such methods are also useful to test the interaction of components of a sample with their naturally occurring binding partners. Components can be tested in membranes in which they exist at nascently low amounts, e.g., native membranes. BSI is particularly useful to perform the assays of this invention as it can detect interactions at very low concentrations and, therefore, provides a very sensitive assay. Examples of analytes can include, but are not limited to, small organic molecules, biopolymers, macromolecular complexes, viruses, and cells.

Accordingly, the interactions can be between antibody-antigen, protein-protein, small molecule-small molecule, small molecule-protein, drug-receptor, antibody-cell, protein-cell, oligonucleotide-cell, carbohydrate-cell, cell-cell, enzyme-substrate, protein-DNA, protein-aptamer, DNA-DNA, RNA-RNA, DNA-RNA, protein-RNA, small molecule-nucleic acid, biomolecule-molecular imprint, biomolecule-protein mimetic, biomolecule-antibody derivatives, lectin-carbohydrate, biomolecule-carbohydrate, small molecule-micelle, small molecule-membrane-bound protein, antibody-membrane-bound protein, or enzyme-substrate. In various aspects, the analyte can be an enzyme or enzyme complex (mixture) which catalyzes the creation of new biomolecules arising from the fusion of biomolecular species (such as a ligase) or replication/amplification of biomolecular species, as is the case in polymerase chain reactions.

Drug candidates useful as analytes in this invention include small organic molecules and biological molecules, i.e., biologics. Organic molecules used as pharmaceuticals generally are small organic molecules typically having a size up to about 500 Da, up to about 2,000 Da, or up to about 10,000 Da. Certain hormones are small organic molecules.

Organic biopolymers can also be used as analytes. Examples of organic biopolymers include, but are not limited to, polypeptides (e.g., oligonucleotides or nucleic acids), carbohydrates, lipids, and molecules that combine these, for example, glycoproteins, glycolipids, and lipoproteins. Certain hormones are biopolymers. Antibodies find increasing use as biological pharmaceuticals. U.S. patent application Ser. No. 11/890,282 provides a list of antibody drugs. This list includes, for example, herceptin, bevacizumab, avastin, erbitux, and synagis (cell adhesion molecules).

Macromolecular complexes also can be used as analytes. They are typically at least 500 Da in size. Examples of macromolecular complexes include, but are not limited to, membrane complexes that are macromolecular assemblies like ion channels and pumps (e.g., Na-K pumps), ATP-ases, secretases, nucleic acid-protein complexes, polyribosomal complexes, polysomes, the p450 complex and enzyme complexes associated with electron transport size.

Viruses and parts of viruses, e.g., capsids and coat proteins, also can be analytes. Cells can be analytes. In this way, for example, cell surface molecules, such as adhesion factors, can be tested. Cells can be, for example, pathogens, cancer cells, inflammatory cells, t-cells, b-cells, NK cells, macrophages, etc.

In a further aspect, the analyte comprises at least one of a small molecule, nucleic acid, polypeptide, carbohydrate, lipid, protein, glycoprotein, lipoprotein, DNA, RNA, DNA-protein construct, or RNA-protein construct. In a still further aspect, the analyte comprises at least one small molecule. In yet a further aspect, the small molecule is a drug candidate.

### F. METHODS OF DETECTING A BINDING INTERACTION

In one aspect, the invention relates to methods of detecting a binding interaction, the method comprising the steps of: a) preparing a sample comprising uncultured tissue homogenate; b) providing an apparatus adapted for performing light scattering interferometry, the apparatus comprising: i) a fluidic device; ii) a channel formed in the fluidic device capable of receiving the sample and an analyte; iii) a light source for generating a light beam; iv) a photodetector for receiving scattered light and generating intensity signals; and v) at least one signal analyzer capable of receiving the intensity signals and determining therefrom the binding interaction between the sample and the analyte; c) introducing the sample and the analyte into the channel; and d) interrogating the sample using light scattering interferometry. In a further aspect, the method further comprises determining one or more characteristic properties of the sample from the intensity signals. In a still further aspect, at least one of the one or more characteristic properties comprises a change in conformation, structure, charge, level of hydration, or a combination thereof.

In one aspect, the invention relates to methods of detecting a binding interaction, the method comprising the steps of: a) preparing a sample comprising uncultured tissue homogenate; b) providing a fluidic device having a channel formed therein for reception of the sample and the analyte; c) introducing the sample and the analyte into the channel; d) directing a light beam from a light source onto the fluidic device such that the light beam is incident on at least a portion of the sample to generate scattered light through reflective and refractive interaction of the light beam with a fluidic device/channel interface, and the sample, wherein the scattered light comprising interference fringe patterns including a plurality of spaced light bands whose positions shift in response to changes in the refractive index of the sample; e) detecting positional shifts in the light bands; and f) determining the binding interaction between the sample and the analyte from the positional shifts of the light bands in the interference fringe patterns. In a further aspect, the method further comprises determining a plurality of characteristic properties of the sample from the interference fringe patterns generated in the channel.

As in conventional BSI, the inventive methods, in one aspect, monitor a change in refractive index to determine the binding affinity of molecular interactions. In such an aspect, the introduction of two binding partners into the channel can create a change in refractive index, resulting in a spatial shift in the generated fringe pattern. In a further aspect, the magnitude of this shift depends on the precise fringes interrogated, the concentration of the binding pairs, conformational changes initiated upon binding, changes in water of hydration, and binding affinity.

When compared to the concentrations and volumes used for ITC and ellipsometry, BSI is 6 orders of magnitude more sensitive than ITC and 8 orders of magnitude more than ellipsometry. This makes BSI interaction-efficient, with the ability to detect a relatively small number of discreet interactions when compared to other free-solution techniques. The simple, user-friendly design of BSI provides a technique by which organic chemists can screen for molecules by following a change in refractive index.

In various aspects, BSI can determine kinetic parameters. That is, the interferometric detection technique described herein can be used to monitor various kinetic parameters, such as, for example, binding affinities, of a chemical and/or biochemical analyte species. The use of BSI for the determination of a kinetic parameter can provide one or more advantages over traditional techniques, for example, free-solution measurements of label-free species, high throughput, small sample volume, high sensitivity, and broad dynamic range. A BSI technique can be performed on a free-solution species, a surface immobilized species, or a combination thereof. In a further aspect, the species of interest is a free-solution species, wherein at least a portion of the species of interest is not bound or otherwise immobilized. In a still further aspect, at least a portion of the species of interest is surface immobilized.

In various aspects, a BSI technique can be used to analyze and/or quantify one or more molecular interactions, such as, for example, a dissociation constant for one or more binding pair species.

The sensitivity of a multiplexed BSI technique can allow analysis and/or determination of at least one kinetic parameter to be performed on a small volume sample. The volume of a sample comprising at least one species of interest can, in various aspects, be less than about 1 nL, for example, about 900, 850, 800, 700, 600, 500, 400, 350, 300, 250, or 200 pL; less than about 600 pL, for example, about 580, 550, 500, 450, 400, 350, 300, 250, or 200 pL; or less than about 400 pL, for example, about 390, 380, 370, 360, 350, 340, 330, 320, 310, 300, 280, 250, 230, or 200 pL. In various aspects, the sample volume is about 500 pL. In a further aspect, the sample volume is about 350 pL. The sample volume can also be greater than or less than the volumes described above, depending on the concentration of a species of interest and the design of a particular BSI apparatus. A species that can be analyzed via BSI can be present in neat form, in diluted form, such as, for example, in a dilute solution, or any other form suitable for analysis by a BSI technique. The concentration of a species of interest can likewise vary depending upon, for example, the design of a particular BSI apparatus, the volume of sample in the optical path, the intensity of a response of a specific species to the radiation used in the experiment. In a still further aspect, the species can be present at a concentration of from about 1 pM to greater than 100 mM.

Analysis of a kinetic parameter via a BSI technique can be performed on a static sample, a flowing sample, for example, 75-120 μL/min, or a combination thereof. In various aspects, analysis of a kinetic parameter via a BSI technique can be performed on a flowing sample having a flow rate of, for example, 10-1,000 nl/min, or less. In a further aspect, an analysis can be a stop-flow determination that can allow an estimation of the dissociation constant (K_{D}) of one or more binding pairs of species. The speed at which one or more samples can be analyzed can be dependent upon, inter alia, the data acquisition and/or processing speed of the detector element and/or processing electronics.

The concentration of one or more analyte species in a sample can be determined with a BSI technique by, for example, monitoring the refractive index of a sample solution comprising an analyte species. A property, such as, for example, refractive index, can be measured in real-time and the kinetics of an interaction between analyte species determined therefrom. Other experimental conditions, such as, for example, temperature and pH, can optionally be controlled during analysis. The number of real-time data points acquired for determination of a kinetic parameter can vary based on, for example, the acquisition rate and the desired precision of a resulting kinetic parameter. The length of time of a specific experiment should be sufficient to allow acquisition of at least the minimal number of data points to calculate and/or determine a kinetic parameter. In various aspects, an experiment can be performed in about 60 seconds.

An apparent binding affinity between binding pair species can subsequently be extracted from the acquired data using conventional kinetics models and/or calculations. In various aspects, a model assumes first order kinetics (a single mode binding) and the observed rate (k_{obs}) can be plotted versus the concentration of one of the species. A desired kinetic parameter, such as, for example, K_{D}, can be determined by, for example, a least squares analysis of the relationship plotted above. A suitable fitting model can be selected based on the particular experimental condition such that a rate approximation can be determined at the end of the analysis. One of skill in the art can readily select an appropriate model or calculation to determine a particular kinetic parameter from data obtained via BSI analysis.

In various aspects, BSI can be utilized to measure a free-solution molecular interaction. In a further aspect, BSI can be used to measure both a free solution property and an immobilized interaction within the same channel. In a still further aspect, BSI can measure label-free molecular interactions.

BSI can be used in any market where measuring macromolecular interactions is desired. In various aspects, a BSI technique, as described herein can be combined with various electrochemical studies. In summary, BSI can be useful as a tool for studying small molecule interactions.

In various aspects, the sample concentration is about equal to the true K_{D} in 0.1% serum. In a further aspect, the sample concentration is about 2 times higher than the true K_{D}. In a still further aspect, the sample concentration is about 3 times higher than the true K_{D}. In yet a further aspect, the sample concentration is about 5 times higher than the true K_{D}. In an even further aspect, the sample concentration is about 10 times higher than the true K_{D}.

In various aspects, the sample concentration is less than the true K_{D} in 0.1% serum. In a further aspect, the sample concentration is about half of the true K_{D}. In a still further aspect, the sample concentration is about one-third of the true K_{D}. In yet a further aspect, the sample concentration is about one-fifth of the true K_{D}. In an even further aspect, the sample concentration is about one-tenth of the true K_{D}.

In a further aspect, the binding interaction is between antibody-antigen, protein-protein, small molecule-small molecule, small molecule-protein, drug-receptor, enzyme-substrate, protein-DNA, protein-aptamer, DNA-DNA, RNA-RNA, DNA-RNA, protein-RNA, small molecule-nucleic acid, biomolecule-molecular imprint, biomolecule-carbohydrate, small molecule-membrane-bound protein, or antibody-membrane-bound protein.

In a further aspect, the sample is mixed with the analyte prior to the introducing step.

In a further aspect, the sample and the analyte are introduced into the channel in label-free solution. In a still further aspect, the concentration of sample in the label-free solution is at least about 10 pM. In yet a further aspect, the concentration of sample in the label-free solution is at least about 1 pM. In an even further aspect, the concentration of sample in the label-free solution is at least about 0.1 pM. In a still further aspect, the concentration of sample in the label-free solution is at least about 0.01 pM. In yet a further aspect, the concentration of sample in the label-free solution is at least about 0.001 pM.

In a further aspect, introducing comprises injecting.

In a further aspect, interrogating comprises monitoring a membrane-associated protein binding event.

In a further aspect, interrogating comprises detecting scattered light on the photodetector, and wherein the scattered light comprises a plurality of interference fringe patterns. In a still further aspect, interrogating comprises detecting back-scattered light on the photodetector, and wherein the back-scattered light comprises a plurality of interference fringe patterns. In yet a further aspect, detecting is under a stop flow configuration. In an even further aspect, detecting is under a flowing configuration. In a still further aspect, the plurality of interference fringe patterns is used to determine the K_{D} of the sample and the analyte.

In a further aspect, the scattered light is incident on a photodetector array.

In a further aspect, the positional shifts in the light bands correspond to a chemical event occurring in the sample. In a still further aspect, the positional shifts in the light bands are used to determine the K_{D} of the sample and the analyte.

### G. METHODS OF DETECTING A BINDING INTERACTION IN MULTIPLE MATRICES

In one aspect, the invention relates to methods of detecting a binding interaction, the method comprising the steps of: a) preparing a first sample comprising a matrix at a first concentration, wherein the matrix is selected from buffer, serum, and/or tissue homogenate; b) preparing a second sample comprising a matrix at a second concentration, wherein the matrix is selected from buffer, serum, and/or tissue homogenate and wherein the matrix of the second sample is different than the matrix of the first sample; c) providing an apparatus adapted for performing light scattering interferometry, the apparatus comprising: i) a fluidic device; ii) a channel formed in the fluidic device capable of receiving the first and/or second sample and an analyte; iii) a light source for generating a light beam; iv) a photodetector for receiving scattered light and generating intensity signals; and v) at least one signal analyzer capable of receiving the intensity signals and determining therefrom the binding interaction between the first and/or second sample and the analyte; d) introducing the first and/or second sample and the analyte into the channel; and e) interrogating the first and/or second sample using light scattering interferometry. In a further aspect, the first concentration is equal to the second concentration.

In various aspects, the first sample comprises buffer at a first concentration and the second sample comprises serum at a second concentration. In a further aspect, the first sample comprises buffer at a first concentration and the second sample comprises tissue homogenate at a second concentration. In a still further aspect, the first sample comprises serum at a first concentration and the second sample comprises tissue homogenate at a second concentration. In yet a further aspect, the tissue homogenate comprises at least one membrane vesicle and/or an interstitial environment. In an even further aspect, the first concentration is equal to the second concentration.

In various aspects, the first concentration is of from about 0.1 wt % to about 100 wt % in aqueous solution. In a further aspect, the first concentration is of from about 0.1 wt % to about 85 wt %. In a still further aspect, the first concentration is of from about 0.1 wt % to about 75 wt %. In yet a further aspect, the first concentration is of from about 0.1 wt % to about 50 wt %. In an even further aspect, the first concentration is of from about 0.1 wt % to about 25 wt %. In a still further aspect, the first concentration is of from about 0.1 wt % to about 10 wt %. In an even further aspect, the first concentration is of from about 10 wt % to about 100 wt %. In a still further aspect, the first concentration is of from about 25 wt % to about 100 wt %. In yet a further aspect, the first concentration is of from about 50 wt % to about 100 wt %. In an even further aspect, the first concentration is of from about 75 wt % to about 100 wt %. In a still further aspect, the first concentration is of from about 85 wt % to about 100 wt %.

In various aspects, the second concentration is of from about 0.1 wt % to about 100 wt % in aqueous solution. In a further aspect, the second concentration is of from about 0.1 wt % to about 85 wt %. In a still further aspect, the second concentration is of from about 0.1 wt % to about 75 wt %. In yet a further aspect, the second concentration is of from about 0.1 wt % to about 50 wt %. In an even further aspect, the second concentration is of from about 0.1 wt % to about 25 wt %. In a still further aspect, the second concentration is of from about 0.1 wt % to about 10 wt %. In an even further aspect, the second concentration is of from about 10 wt % to about 100 wt %. In a still further aspect, the second concentration is of from about 25 wt % to about 100 wt %. In yet a further aspect, the second concentration is of from about 50 wt % to about 100 wt %. In an even further aspect, the second concentration is of from about 75 wt % to about 100 wt %. In a still further aspect, the second concentration is of from about 85 wt % to about 100 wt %.

In a further aspect, the first and/or second sample is mixed with the analyte prior to the introducing step. In a still further aspect, the first sample is mixed with the analyte prior to the introducing step. In yet a further aspect, the second sample is mixed with the analyte prior to the introducing step. In an even further aspect, the first and the second sample are mixed with the analyte prior to the introducing step.

In a further aspect, interrogating comprises detecting scattered light on the photodetector, and wherein the scattered light comprises a plurality of interference fringe patterns. In a still further aspect, interrogating comprises detecting back-scattered light on the photodetector, and wherein the back-scattered light comprises a plurality of interference fringe patterns. In yet a further aspect, the plurality of interference fringe patterns is used to determine the K_{D} of the first and/or second sample and the analyte.

In a further aspect, the method further comprises generating a plot of sample concentration versus the K_{D} value for the first and second sample.

### H. METHODS OF DETECTING A BINDING INTERACTION USING MULTIPLE CONCENTRATIONS

In one aspect, the invention relates to a method of detecting a binding interaction, the method comprising the steps of: a) preparing a first sample comprising a matrix at a first concentration, wherein the matrix is selected from buffer, serum, and/or tissue homogenate; b) preparing a second sample comprising a matrix at a second concentration, wherein the matrix is selected from buffer, serum, and/or tissue homogenate, and wherein the matrix of the second sample is the same as the matrix of the first sample; c) providing an apparatus adapted for performing light scattering interferometry, the apparatus comprising: i) a fluidic device; ii) a channel formed in the fluidic device capable of receiving the first and/or second sample and an analyte; iii) a light source for generating a light beam; iv) a photodetector for receiving scattered light and generating intensity signals; and v) at least one signal analyzer capable of receiving the intensity signals and determining therefrom the binding interaction between the first and/or second sample and the analyte; d) introducing the first and/or second sample and the analyte into the channel; and e) interrogating the first and/or second sample using light scattering interferometry.

In a further aspect, the first concentration is not equal to the second concentration. In a still further aspect, the first concentration is greater than the second concentration. In yet a further aspect, the first concentration is less than the second concentration.

In various aspects, the first concentration is of from about 0.1 wt % to about 100 wt % in aqueous solution. In a further aspect, the first concentration is of from about 0.1 wt % to about 85 wt %. In a still further aspect, the first concentration is of from about 0.1 wt % to about 75 wt %. In yet a further aspect, the first concentration is of from about 0.1 wt % to about 50 wt %. In an even further aspect, the first concentration is of from about 0.1 wt % to about 25 wt %. In a still further aspect, the first concentration is of from about 0.1 wt % to about 10 wt %. In an even further aspect, the first concentration is of from about 10 wt % to about 100 wt %. In a still further aspect, the first concentration is of from about 25 wt % to about 100 wt %. In yet a further aspect, the first concentration is of from about 50 wt % to about 100 wt %. In an even further aspect, the first concentration is of from about 75 wt % to about 100 wt %. In a still further aspect, the first concentration is of from about 85 wt % to about 100 wt %.

In various aspects, the second concentration is of from about 0.1 wt % to about 100 wt % in aqueous solution. In a further aspect, the second concentration is of from about 0.1 wt % to about 85 wt %. In a still further aspect, the second concentration is of from about 0.1 wt % to about 75 wt %. In yet a further aspect, the second concentration is of from about 0.1 wt % to about 50 wt %. In an even further aspect, the second concentration is of from about 0.1 wt % to about 25 wt %. In a still further aspect, the second concentration is of from about 0.1 wt % to about 10 wt %. In an even further aspect, the second concentration is of from about 10 wt % to about 100 wt %. In a still further aspect, the second concentration is of from about 25 wt % to about 100 wt %. In yet a further aspect, the second concentration is of from about 50 wt % to about 100 wt %. In an even further aspect, the second concentration is of from about 75 wt % to about 100 wt %. In a still further aspect, the second concentration is of from about 85 wt % to about 100 wt %.

In various aspects, the concentration of the first and/or second sample is about 10 times higher than the true K_{D}. In a further aspect, the concentration of the first and/or second sample is about 20 times higher than the true K_{D}. In a still further aspect, the concentration of the first and/or second sample is about 30 times higher than the true K_{D}. In yet a further aspect, the concentration of the first and/or second sample is about 40 times higher than the true K_{D}. In an even further aspect, the concentration of the first and/or second sample is about 50 times higher than the true K_{D}.

In various aspects, the first sample comprises buffer at a first concentration and the second sample comprises buffer at a second concentration. In a further aspect, the first sample comprises serum at a first concentration and the second sample comprises serum at a second concentration. In a still further aspect, the first sample comprises tissue homogenate at a first concentration and the second sample comprises tissue homogenate at a second concentration. In yet a further aspect, the tissue homogenate comprises at least one membrane vesicle and/or an interstitial environment.

In a further aspect, the first and/or second sample is mixed with the analyte prior to the introducing step. In a still further aspect, the first sample is mixed with the analyte prior to the introducing step. In yet a further aspect, the second sample is mixed with the analyte prior to the introducing step. In an even further aspect, the first and the second sample are mixed with the analyte prior to the introducing step.

In a further aspect, interrogating comprises detecting scattered light on the photodetector, and wherein the scattered light comprises a plurality of interference fringe patterns. In a still further aspect, interrogating comprises detecting back-scattered light on the photodetector, and wherein the back-scattered light comprises a plurality of interference fringe patterns. In yet a further aspect, the plurality of interference fringe patterns is used to determine the K_{D} of the first and/or second sample and the analyte. In an even further aspect, the K_{D} of the first and/or second sample and the analyte is right-shifted.

In a further aspect, the method further comprises generating a plot of sample concentration versus the K_{D} value for the first and second sample.

### I. METHODS OF PREDICTING THE IN VIVO BINDING AFFINITY

In one aspect, the invention relates to methods of predicting the in vivo binding affinity of an analyte, the method comprising the steps of: a) preparing a sample comprising uncultured tissue homogenate; b) providing a fluidic device having a channel formed therein for reception of the sample and the analyte; c) introducing the sample and an analyte into the channel; d) directing a light beam from a light source onto the fluidic device such that the light beam is incident on at least a portion of the sample to generate scattered light through reflective and refractive interaction of the light beam with a fluidic device/channel interface, and the sample, wherein the scattered light comprising interference fringe patterns including a plurality of spaced light bands whose positions shift in response to changes in the refractive index of the sample; e) detecting positional shifts in the light bands; f) determining the K_{D} of the sample and the analyte using the positional shifts in the light bands; and g) predicting the in vivo behavior using the binding affinity.

Molecular interactions govern biology, human health, disease, and the pharmacological efficacy of therapeutics (both small molecules and biologics). Therapeutic dose-response relationships are predicated upon accurate measures of drug binding interactions to a target at the site of action. Clinically relevant measurements are especially problematic since target proteins reside in complex physiological environments, such as biological fluids, or tissue microenvironments as soluble and/or membrane-bound forms.

Thus, in various aspects, the invention relates to methods of predicting the in vivo binding affinity of an analyte, the method comprising using light scattering interferometry to measure K_{d} values for soluble target and membrane-bound target independently. In a further aspect, the light scattering interferometry simultaneously measures integrated K_{d} to membrane-bound target bathed in soluble target, thereby mimicking the tissue and interstitial environment.

### J. KITS

In various aspects, the invention relates to kits comprising the disclosed apparatus, a sample comprising uncultured tissue homogenate, and one or more of: a) an analyte; b) a sample comprising at least one membrane vesicle; c) a sample comprising serum; d) a sample comprising buffer; e) instructions for interrogating a sample; f) instructions for detecting a binding interaction; and g) instructions for predicting the in vivo binding affinity of the analyte.

It is contemplated that the disclosed kits can be used in connection with the disclosed methods of preparing, the disclosed methods of detecting and/or the disclosed methods of predicting.

### K. DIAGNOSTIC AND THERAPEUTIC USES

The disclosed methods are especially useful when employed in connections with diagnostic methods and/or therapy tracking. More specifically, the detection step of the disclosed methods can be used as a replacement for the detection step in conventional diagnostic methods.

In one specific aspect, the disclosed methods can be used in connection with Enzyme-Linked Immunosorbant Assays (ELISA). For example, the detection step of the disclosed methods can be used as a replacement for conventional detections steps (e.g., fluorescence, luminescence, etc.) in ELISA.

### L. EXAMPLES

Realizing success for new molecularly targeted therapeutics requires early in-vitro/in-vivo correlation (IVIVC) for clinical implementation. Drug candidates need to be confidently profiled for pharmacokinetics/pharmacodynamics (PKPD) to avoid costly downstream attrition. Precise, intrinsic potency estimations have been confounded by the inability to account for molecular dynamics, systems physiology, disease pathology and adequate target exposure. Herein, in vitro dose response curves across increasingly complex matrices are used to provide a refined, contextual assessment for clinical modeling. Ensemble binding affinities gave excellent correlation to human data. Given the intense political discourse on health care budgets, cost-effective proof of concept for new drugs necessitates more complete taxonomy modeling. Interactome-centric conditions for pharmacologic measurements, as demonstrated herein using backscattering interferometry (BSI), produce reliable dose response curves that will enable more accurate first-in-man dose estimations. To rapidly and easily probe a protein's quinary structure, within the context of its' complex network, could be the “Indra's net” for drug discovery.

Indra's net is a concept portraying how a jewel at each vertex of a net provides a reflection of every strand convergence in the network. This metaphor is used to illustrate that accounting for biological multidimensionality would provide more physiologically estimations for dosing. Given the diminished harvest of drugs in recent decades, often attributable to lack of efficacy (30%) and/or toxicity (20%) (Kola, I. and Landis, J. (2004) Nat. Rev. Drug Discov. 3, 711-715), a more accurate estimate of target coverage to predict human dosing and therapeutic index (TI) that could reduce late-stage attrition. This disparity is likely due to both lack of contextual data and limited sensitivity of platforms capable of probing the full interactome (i.e., an inadequate net) (Araujo, R. P., et al. (2007) Nat. Rev. Drug Discov. 6, 871-880).

Drugs diffuse across matrices toward targets in complex physiologic environments. Free solution, label-free BSI binding assays can capture both the biodiversity of target environments and the complexity of binding scenarios (Baksh, M. M., et al. (2011) Nat. Biotechnol. 29, 357-360. Additionally, pharmacokinetics and tissue distribution studies have been described for mAbs to quantify drug exposure at the site of action. Drug binding to target at the site of action and target concentrations, assures interaction or coverage of that target. Local and systemic target concentrations are determined by rates of synthesis/degradation unique to each protein target and physiological state (Fernandez Ocana, M., et al. (2012) Analytical Chemistry 84, 5959-5967. The data herein was generated in conditions permitting the natural/physiologic state of targets while accounting for matrix effects and off-site binding. A range of K_{d} values were generated, from simple solution to tissue, acknowledging biological/physiological “quantum entanglement.” This platform casts a much wider net for harvesting data.

Soluble target, protein conformation, variation of target concentrations across matrices, and native environment were all taken into account when measuring binding affinity of PF-00547659, a fully human anti-IgG2 monoclonal antibody (mAb) for anti-human mucosal addressin cell adhesion molecule (MAdCAM). MAdCAM is an important therapeutic target, expressed as both a soluble and a trans-membrane protein, that mediates either rolling or firm adhesion of lymphocytes via integrin α4β7⁺, to specialized high endothelial vessels (Pullen, N., et al. (2009) Br. J. Pharmacol. 157, 281-293. PF-00547659 was developed to treat inflammatory bowel disease (IBD) and has been shown to reduce mucosal damage in animal models of colitis (Apostolaki, M., et al. (2008) Gastroenterology 134, 2025-2035; Hokari, R., et al. (2001) Clin. Exp. Immunol. 126, 259-265; Goto, A., et al. (2006) Inflamm. Bowel Dis. 12, 758-765). Soluble MAdCAM has been measured in the serum and urine of healthy subjects and in the synovium of osteoarthritis patients, while membrane-bound protein is constitutively expressed immune tissue including the small intestine (Leung, E., et al. (2004) Immunol. Cell Biol. 82, 400-409). Incongruence in PF-00547659/MAdCAM binding measurements across platforms and matrices led to the development of eTCM/eK_{d} to provide a more accurate “net” value.

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

1. Experimental Methods

Examples disclosed herein illustrate the invention's utility but do not limit the scope invention scope.

a. Materials

Soluble human MAdCAM-IgG1 Fc fusion protein, CHO cells stably expressing full length hMAdCAM and PF-00547659, a fully human anti-MAdCAM IgG2 monoclonal antibody (mAb) were generated internally as described previously by Pullen et al. (Pullen, N., et al. (2009) Br. J. Pharmacol. 157, 281-293). The human serum that was pooled from 6 to 8 donors was purchased from Bioreclamation.

a. Vesicle Preparation

The vesicles were prepared from both CHO cells stably expressing full length hMAdCAM and colon tissues from patients with Ulcerative Colitis. The colon tissues were homogenized as described herein. Cells were incubated in a hypotonic solution, gently lysed, and the internal components separated from the outer membranes by centrifugation. Outer membranes were then sonicated and centrifuged to create a uniform population of small unilamellar vesicles containing native proteins.

b. Vesicle-Rich Homogenate (VRH) Preparation

Approximately 50 mg of colon tissue was placed in a 15 mL tube using a scalpel and petri dish. The tube was then stored in ice until the sample was sufficiently thawed, before being placed into a cold mortar and pestle, covered in ˜500 μL of sonication buffer comprising PBS with 2× protease inhibitor, and ground until homogenous. The homogenized tissue was then transferred into a 1.6 mL centrifuge tube. Additionally, the mortar was rinsed with an additional 500 μL of sonication buffer, which was then added to the centrifuge tube. The centrifuge tube was then vortexed for several seconds on a medium setting. The solution was sonicated using a dram glass vial for ˜2 minutes (pulsed 5 sec. on/1 sec. off) before being transferred into a centrifuge tube and centrifuged at 4° C. and 8,000 xg for 1 hour. At this time a substantial pellet had formed, which was removed from the supernatant. The supernatant was diluted with an additional 2 mL of cold sonication buffer.

Dynamic light scattering was then used to check the size and PDI of the tissue vesicles. When the size was found to be too large (>200d·nm) or the polydispersity high (>0.30), the vesicles were sonicated for an additional hour. Once the vesicles were deemed acceptable via DLS, the Bradford Reagent was used to determine protein content.

c. Interstitial-Like Homogenate (ILH) Preparation

Colon tissue from healthy volunteer was homogenized in 1:4 (w:v) of PBS with 1× protease inhibitor (from thermo, prod#78430, no EDTA) using Bullet Blender Storm according to the manufacturer's manual. After the sample was centrifuged at 2000×g for 10 minutes, the supernatants were taken out and snap frozen in liquid nitrogen for further experiments.

d. Concentration Measurements of hMAdCAm in Biological Samples

LC-MS/MS based methods were employed for the quantitation of human MAdCAM in vesicles from CHO cells and human colon tissue vesicles and to determine hMAdCAM levels in serum and healthy human colon homogenate. The employed assays targeted a unique, proteotypic peptide sequence from the extracellular domain of the receptor that was enzymatically generated using trypsin as part of the assay procedure. This target peptide and a corresponding stable isotope labeled peptide standard were enriched using an anti-peptide antibody prior to LC-MS/MS. The workflow for processing of vesicles involved acetone precipitation to pellet proteins, whilst serum proteins were denatured in-solution using urea. Subsequently, both protocols entailed reduction of disulfide bonds and alkylation of cysteine residues prior to trypsin digestion.

2. Tissue Preparation

Tissue from male or female subject (preclinical or clinical), normal, pathological or deceased are sources of tissue. One slice of tissue (˜50 micrometer in thickness or 50 microgram in weight) is sufficient to run BSI tissue Kd measurements. These tissues can be obtained through biopsy or from an encapsulated end wedge removed from patients undergoing resection for removal of, for example liver tumors or from resected segments from whole tissue such as livers obtained from multi-organ donors. In contrast to establishing primary cells from the amount of cells from a biopsy would often not be enough to prepare a primary cell culture. See ATCC primary cell culture guide. See also Godoy, P., et al. (2013) “Recent advances in 2D and 3D in vitro systems using primary hepatocytes, alternative hepatocyte sources and non-parenchymal liver cells and their use in investigating mechanisms of hepatotoxicity, cell signaling and ADME.” Archives of Toxicology 87, 1315-1530.

Tissue is comprised of parenchyma cells, and non-parenchyma cells (NPCs). Take liver, a widely used organ for primary cell culture, for example, the non-parenchyma cells include NPCs such as stellate cells of the connective tissue, endothelial cells of the sinusoids, Kupffer cells and immune cells, such as lymphocytes (T cells, B cells, natural killer (NK) and especially NKt cells) and leukocytes. When a tissue homogenate/vesicle is prepared, all representative cell types are present from the ex vivo original tissue sample. In contrast, most of the current activities in developing primary liver cell culture focuses on the parenchymal cell, the hepatocyte itself and the non-parenchyma cells were not present in the culture system. In addition, because different cell types grow and divide at different rates, die or do not grow or divide at all, in culture, culturing a mixed cell population in vitro results in some cell types over growing and dominating the culture, thus increasing the variability of the sample, reducing the consistency of sampling and not representing the original cell milieu present in the original sample obtained from the subject. Sampling directly from tissue removes these variables and maintains all cell types (localized to the organ region, vasculature, etc.), soluble proteins, interstitial fluid, non-cellular tissue components (e.g., fat, collagen, etc.). Godoy, P., et al., supra.

The tissue homogenate/vesicles preparation process involves the mildest mechanical forces to disrupt the cell junctions, whereas collagenase, elastase, DNAase and/or hyaluronidase enzymes are required to break up interconnecting collagen structures to release cells and be able to propagate in culture as primary cells. ATCC primary cell culture guide. In addition, with each passage of primary cells obtained from tissue the protein expression pattern can change dramatically due to the lack of contact signals present within tissue and close proximity of dependent tissue layers. Godoy, P., et al., supra.

3. Binding Isotherms Assays Using Recombinant, Human Fusion Protein

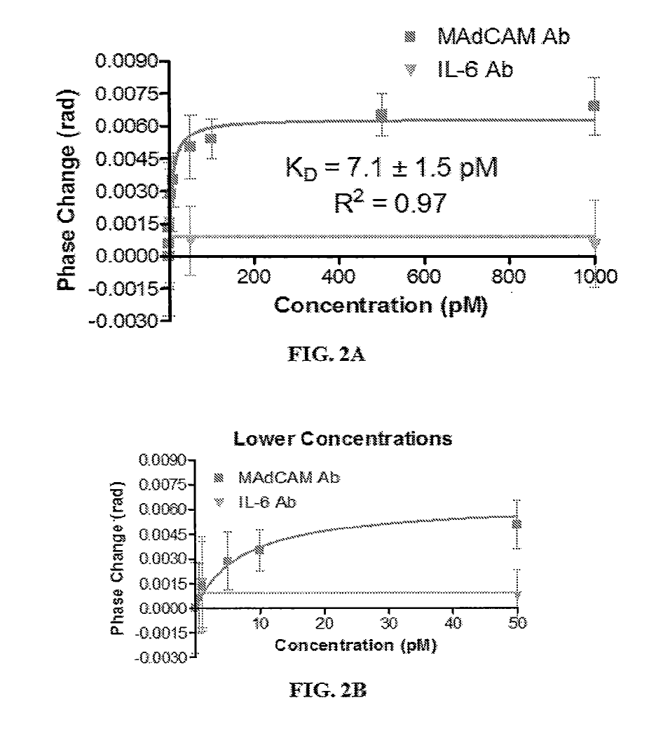

Binding isotherms assays were performed under equilibrium conditions using BSI and compared to the widely used, label-free assay Surface Plasmon Resonance (SPR) (Pullen, N, et al. (2009) Br. J. Pharmacol. 157, 281-293). Using the same recombinant, human fusion protein, with both binding partners untethered in buffer, a K_{d} of 7.1 (±1.5) pM was measured (FIGS. 2A and 2B), a value close to the SPR result (16.1 pM) (Table 1). The small discrepancy between these two values is likely due to the free-solution conditions that do not perturb the measurement (Olmstead, I. R., et al. (2012) Analytical Chemistry 84, 10817-10822). To more closely approximate native conditions, 0.1% healthy human serum was used to measure binding to endogenous, shed, soluble MAdCAM and a K_{d} of 7.5 (±1.5) pM was calculated. These results are meaningful because the protein concentrations in solution here were kept constant at 10 and 1 pM, (Table 2) a concentration where other platforms perform poorly (Kastritis, E., et al. (2011) Clin. Lymphoma Myeloma Leuk. 11, 127-129). In addition, the experimental conditions were at protein concentrations at or below the K_{d} value, requisite for performing accurate or “true” K_{d} measurements (Lowe, P. J., et al. (2009) Basic Clin. Pharmacol. Toxicol. 106, 195-209; Chang, K. J., et al. (1975) Biochim Biophys. Acta 406, 294-303).

TABLE 1

| | | | Target | K_{D} |
|---|---|---|---|---|
| Reference | Method | Matrix | Form | (pM) |
| Pullen, N, et al. (2009) | Biacore | Buffer | Rhu | 16.1 |
| Br. J. Pharmacol. 157, | | | MAdCAM.Fc, | |
| 281-293 | | | soluble | |
| Martin (2009), derived | Clinical | Serum | Endogenous, | 528 |
| from fitting PK/PD | | | soluble | |
| (total MAdCAM serum | | | | |
| concentrations) data to a | | | | |
| TMDD model | | | | |

TABLE 2

| | Target Concentration | K_{d} | |
|---|---|---|---|
| Target | (pM) | (pM) | R² |
| rhMAdCAM-IgG1 | 10 | 7.1 ± 1.5 | 0.97 |
| Fc fusion protein | | | |
| 0.1% normal human | 1 | 7.5 ± 1.5 | 0.98 |
| serum pool | | | |
| CHO-rhMAdCAM | 34 | 134 ± 41 | 0.95 |
| vesicle preparation | | | |
| Human IBD colon | 0.045 | 155 ± 41 | 0.97 |
| vesicle preparation | | | |

4. In Vitro Affinity of MAdCAM Ab Binding to MAdCAM in Increasing Concentrations of Serum

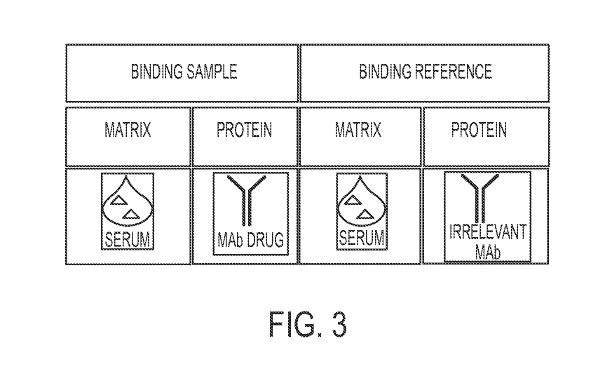

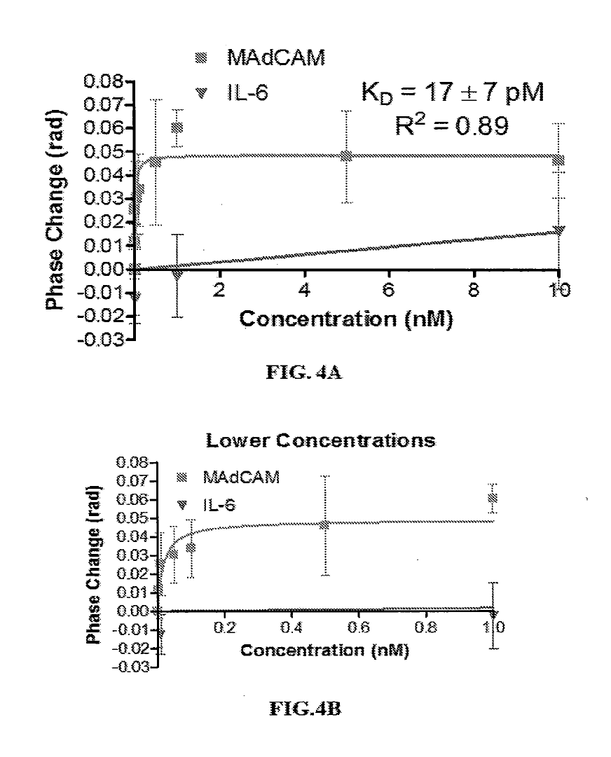

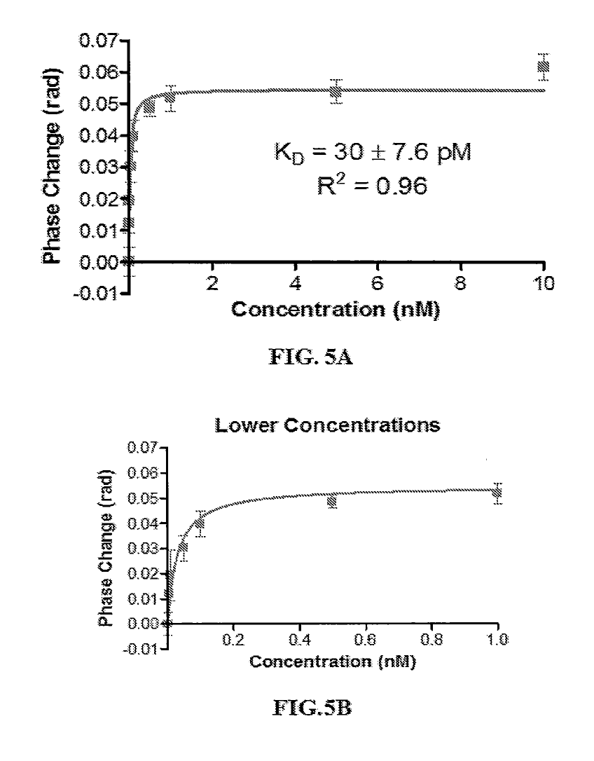

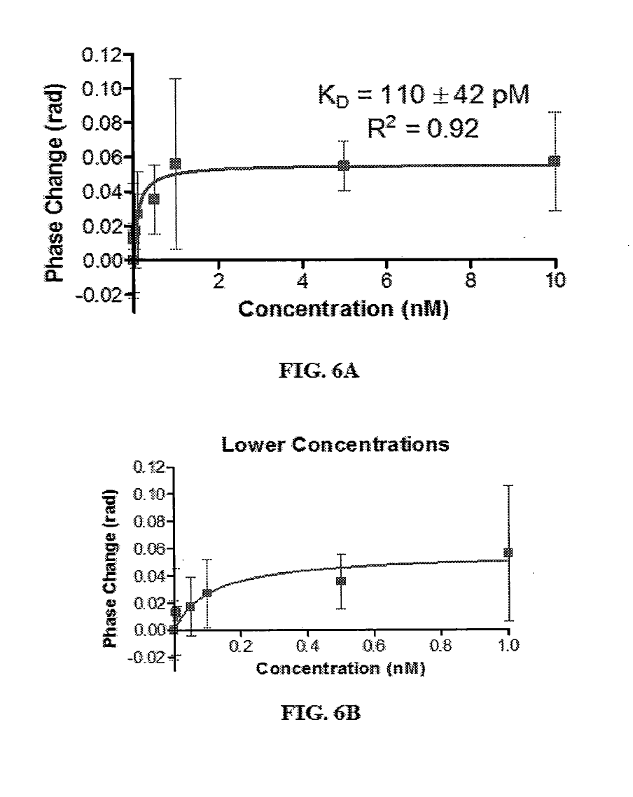

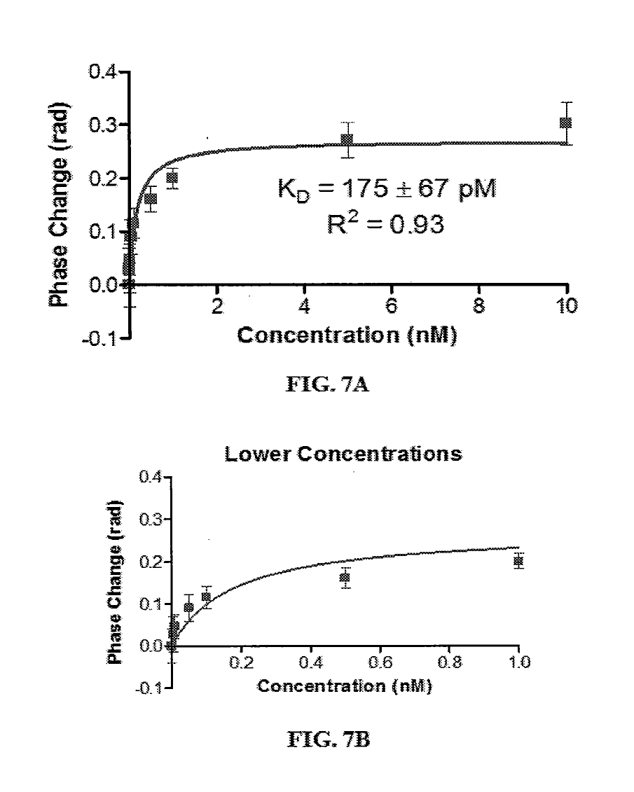

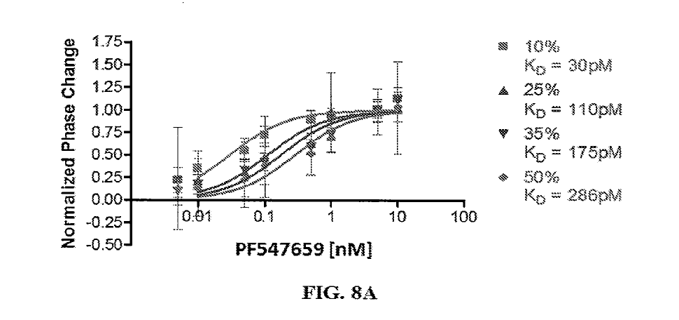

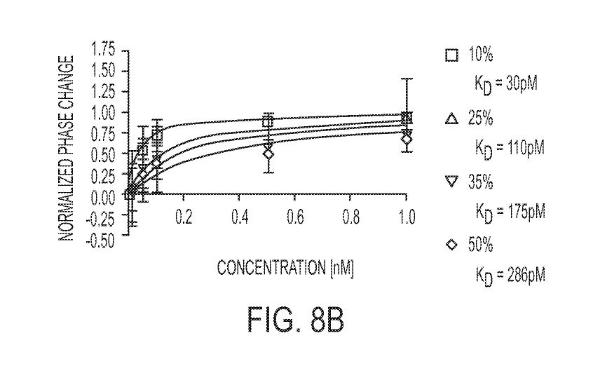

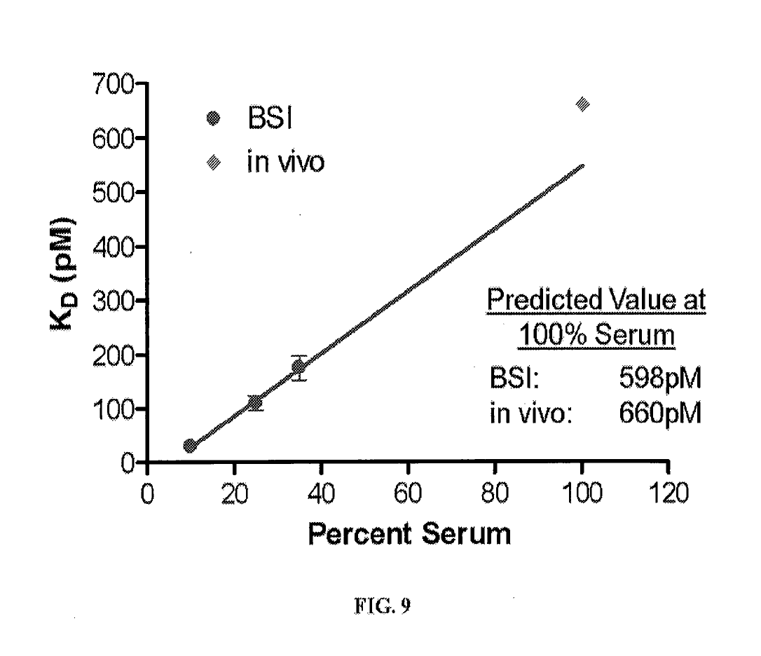

To appraise how an even more relevant and complex native matrix affects binding, affinity measurements were extended to increasing concentrations of endogenous MAdCAM in serum (see FIG. 3 for the BSI experimental set-up). Ligand-binding assays were performed in pooled normal human serum (from Biroreclamation) of 10%, 25%, 35% and 50%. Isotype-matched, anti-IL6 mAb served as an irrelevant control where endogenous IL6 in normal human serum is at physiologic concentrations of 5 pg/mL (Robak, T., et al. (1998) Mediators Inflamm. 7, 347-353). Receptor-ligand dissociation constants are conventionally calculated in conditions where target concentration is equal to or less than the K_{d} value. The portion of linearity in the binding curve increases as receptor concentration increases, relative to the true K_{d} value, a relationship first described by Chang et al. (Chang, K. J., et al. (1975) Biochim. Biophys. Acta. 406, 294-303). Predictably, right-shifted K_{d} values of 30 (FIGS. 5A and 5B), 110 (FIGS. 6A and 6B), 174 (FIGS. 7A and 7B), and 285 pM were observed (Table 3 and 8). By plotting the apparent K_{d} values versus the serum MAdCAM concentration (100, 250, 350 and 500 pM, as determined by LCMS), a linear relationship (r²=0.97) was obtained. Extrapolating to a value of 100% serum gave an estimated apparent Kₐ of 598 pM (FIG. 9). This value correlates well with the Target Mediated Drug Disposition (TMDD) modeling, clinically derived data of 528 pM (see Table 1; Martin 2009). Under these conditions, hMAdCAM concentrations in serum were 10-50 times higher than the true K_{d} of the receptor/ligand pair. Thus, when the apparent and physiologically relevant K_{d} is measured using BSI, it is right-shifted and approximates the clinically derived K_{d}.

TABLE 3

| Normal hu- | Soluble target | Apparent | |
|---|---|---|---|
| man serum | concentration^{a} | K_{d} | |
| (%) | (pM) | (pM) | R² |
| 10 | 100 | 30 ± 7.6 | 0.96 |
| 25 | 250 | 110 ± 41 | 0.92 |
| 35 | 350 | 174 ± 66 | 0.93 |
| 50 | 500 | 285 ± 103 | 0.94 |

^{a}measured by LC-MS/MS.

In the drug development process, an in vivo K_{d} value can sometimes be derived pre-clinically or clinically, based on drug or target concentrations in serum. However, when the drug target is membrane-bound and the source of expression is from tissues, it becomes very difficult to acquire a K_{d} value, which may be different from the K_{d} interacting with soluble target and may be more important in driving efficacy.

5. In Vitro Affinity of MAdCAM Ab Binding to MAdCAM in Cell Vesicles

Modeling efforts and crystal structures have revealed the biologically relevant surface structure where PF-00547659 binds. This integrin-binding (D₁) loop of MAdCAM was shown to be unusually and inherently flexible (Yu, Y., et al. (2013) The Journal of biological chemistry 288, 6284-6294). Therefore, conformational mobility (Yu, Y., et al. (2012) The Journal of cell biology 196, 131-146), as well as the potential for oligermerization (Dando, J., et al. (2002) Acta crystallographica. Section D, Biological crystallography 58, 233-241) necessitate a trans-membrane environment to measure whether differences in affinity between binding to soluble versus membrane-bound protein exist. In order to mimic the original trans-membrane orientation, rather than construct an inauthentic lipid membrane, cell vesicles from CHO cell pellets were generated (Baksh, M. M., et al. (2011) Nat. Biotechnol. 29, 357-360). Briefly, cells stably over-expressing full-length hMAdCAM (Pullen, N. et al. (2009) Br. J. Pharmacol. 157, 281-293) were subjected to hypotonic lysis in PBS with protease inhibitor (2×). The pellet was re-suspended in buffer and sonicated on ice, in a pulsed fashion. The suspension was then centrifuged at 10,000 g for 1 hour at 4° C. The pellet was recovered and characterized for particle size of approximately 115 nm in diameter, and target receptor concentration was quantified (see Table 2). Without wishing to be bound by theory, this environment may account for the increased complexity of the membrane environment that impacts protein conformation, topology, and membrane-matrix interactions (including potential receptor internalization).

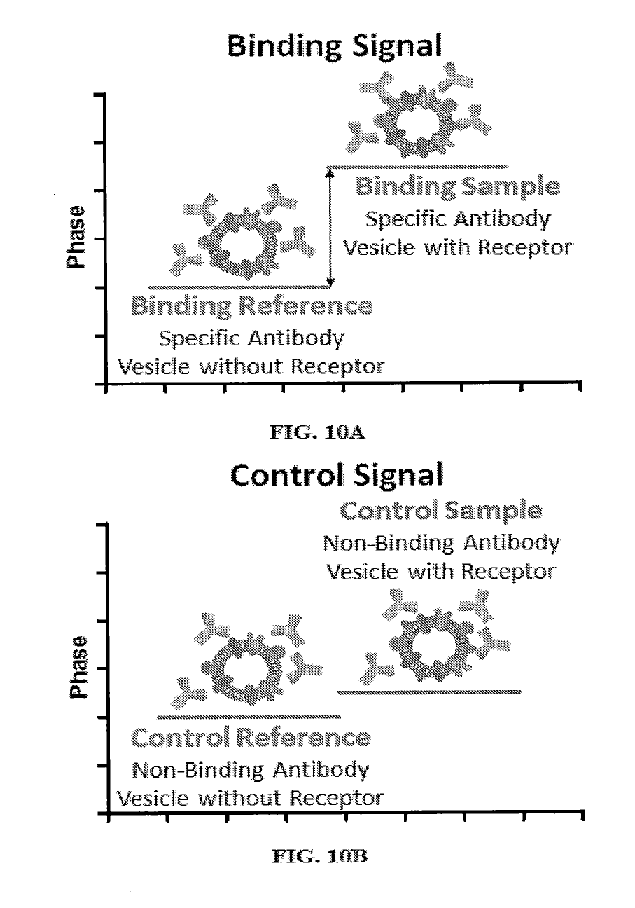

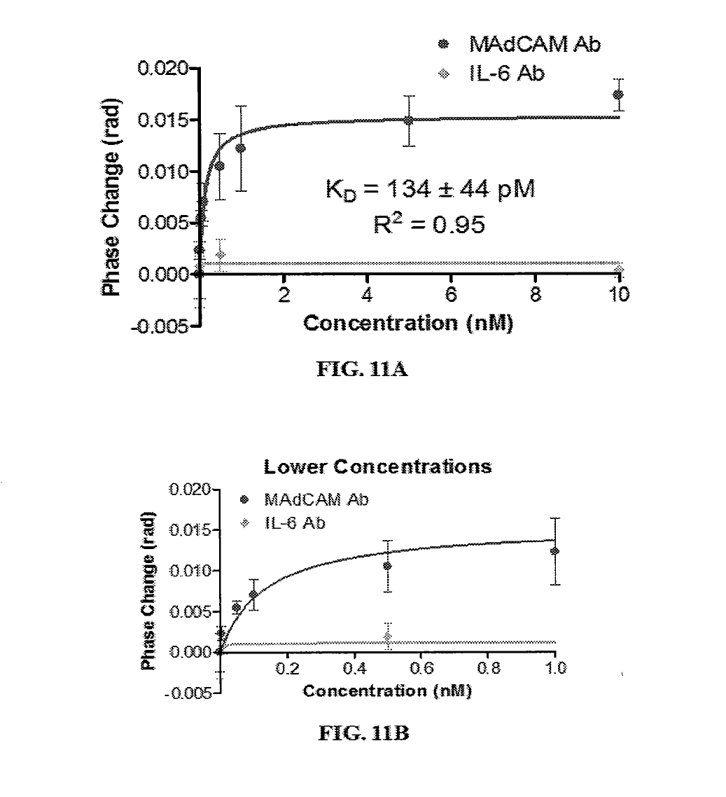

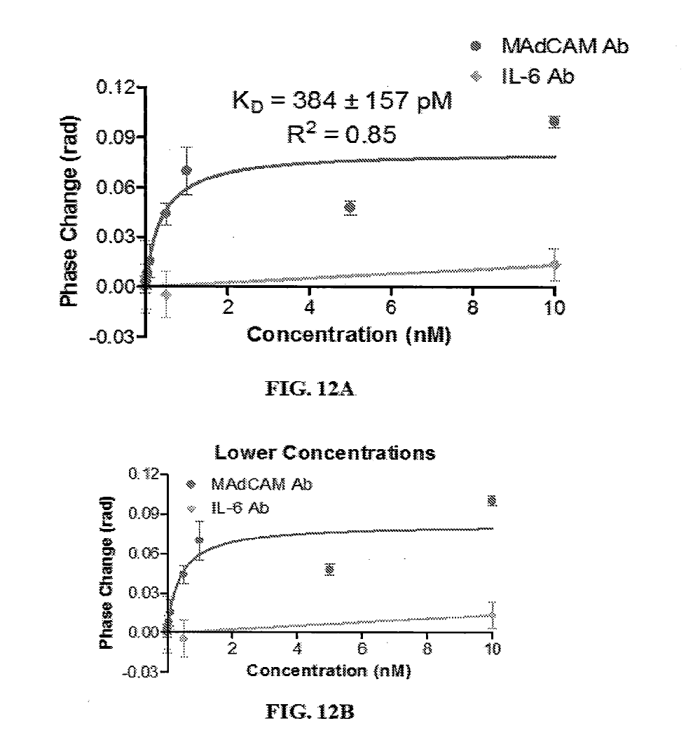

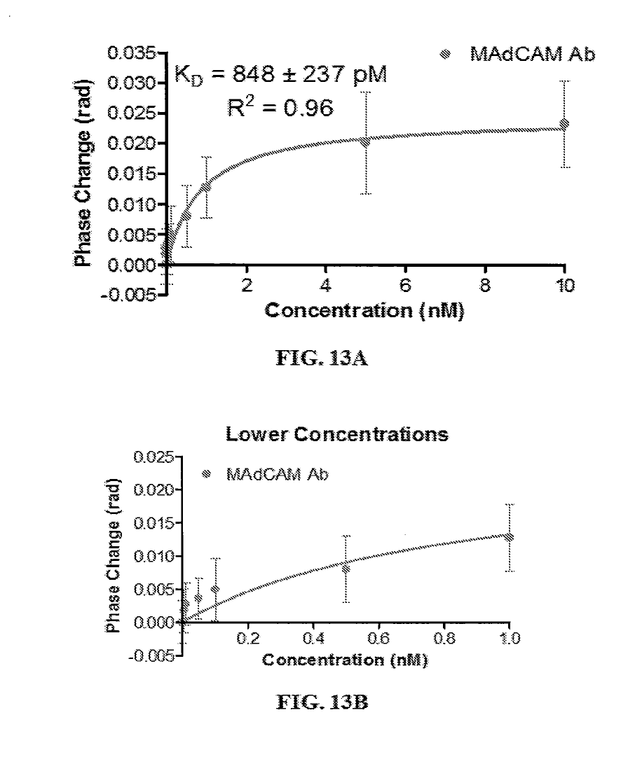

The experimental design is depicted in FIGS. 10A and 10B. Background was subtracted from the signal using wild type (wt) vesicles+PF-00547659 as binding pairs, (no or non-specific binding). Here, in a habitat mimicking the true membrane, protein MAdCAM concentration was 34 pM and a K_{d} of 134 pM was measured in PBS (FIGS. 11A and 11B; see FIGS. 12A and 12B for K_{d} measured in 25% serum and FIGS. 13A and 13B for K_{d} measured in 25% tissue homogenate). While the protein concentration here is ˜4.8-fold higher than the “true” Kₐ measured in 0.1% serum, theory predicts that the relative “error” of this measurement would be no greater than 5-fold of the K_{d} (Chang, K. J., et al. (1975) Biochim Biophys Acta 406, 294-303). However, a K_{d} that is ˜20-fold greater is obtained. This indicates that the binding mechanism is environment-driven and grossly affected by the lipid bi-layer. Without wishing to be bound by theory, the anchoring and conformation restrictions of membrane-bound protein may decrease the affinity of the drug compared to soluble MAdCAM (Schiller, H. B. and Fassler, R. (2013) EMBO reports 14, 509-519). BSI signal has been previously shown to be a function of conformation and hydration changes upon binding (Bornhop, D. J., et al. (2007) Science 317, 1732-1736; Adams, N. M., et al. (2013) Nucleic acids research 41, e103). The use of recombinant, overexpressed, human MAdCAM protein and non-native cell type may not be translatable data to humans; therefore this experiment laid the foundation for testing a more reliable mimic of human disease. To dive even deeper into revealing the nature of the elusive clinical K_{d} necessitates obtaining measurements from a more pertinent ensemble: human tissue consisting of cell vesicles and the microenvironment.

6. In Vitro Affinity of MAdCAM Ab Binding to MAdCAM in Vesicle Rich Homogenate (VRH)

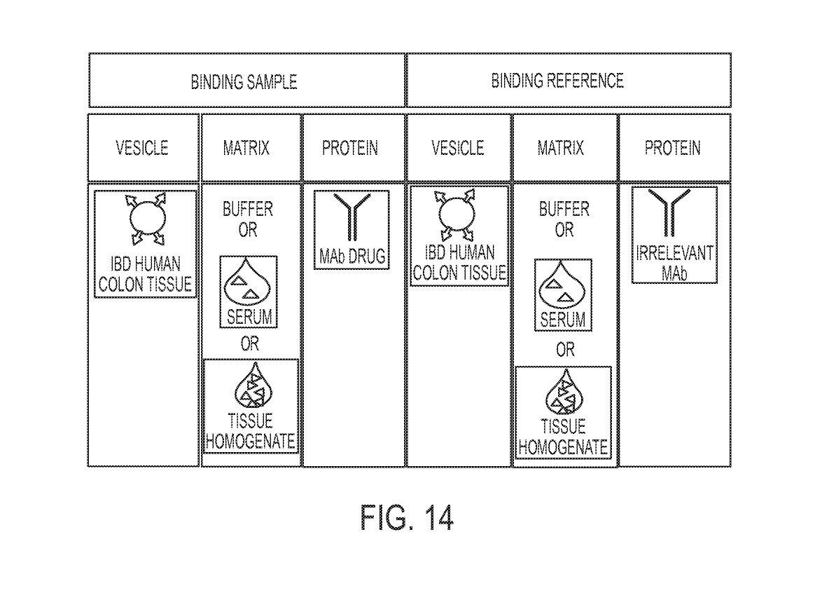

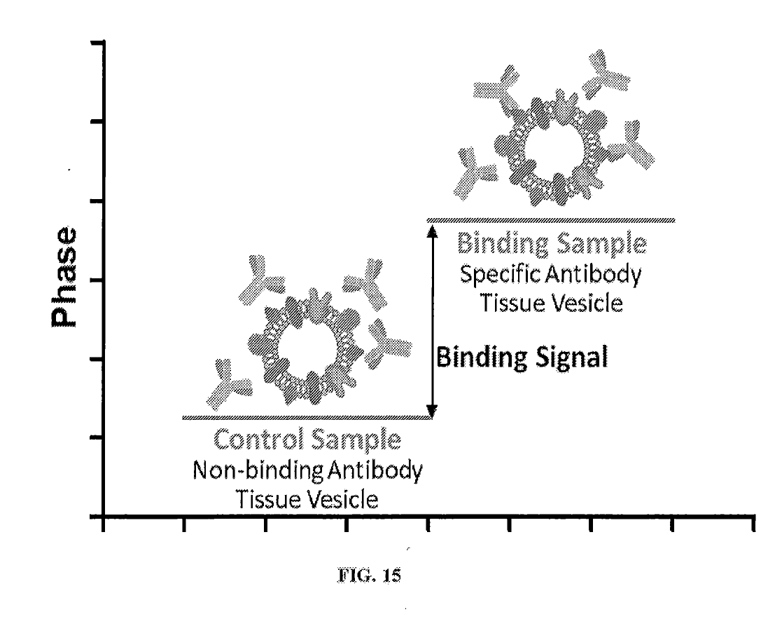

Obtaining a K_{d} value from tissue is problematic due to the limited accessibility of such samples and the constraints of existing assay methodologies (Kastritis, E., et al. (2011) Clin. Lymphoma Myeloma Leuk. 11, 127-129). To obtain an estimate of the affinity of PF-00547659 to membrane-bound MAdCAM in human colon, vesicles were generated from ex-vivo tissue of patients with ulcerative colitis (UC). VRH is a source of cell membrane, protein, growth factors, and cytoskeleton components, thereby providing a more authentic model for simulating the biologic complexity of the human colon (FIG. 14). Briefly, homogenate was prepared similarly to the CHO cell vesicles, but homogenation was with a mortar and pestle. The solution was centrifuged at 10,000 g for 1 hour at 4° C., and the supernatant collected for analysis. Isotype-matched, anti-IL6 mAb served as an irrelevant control.

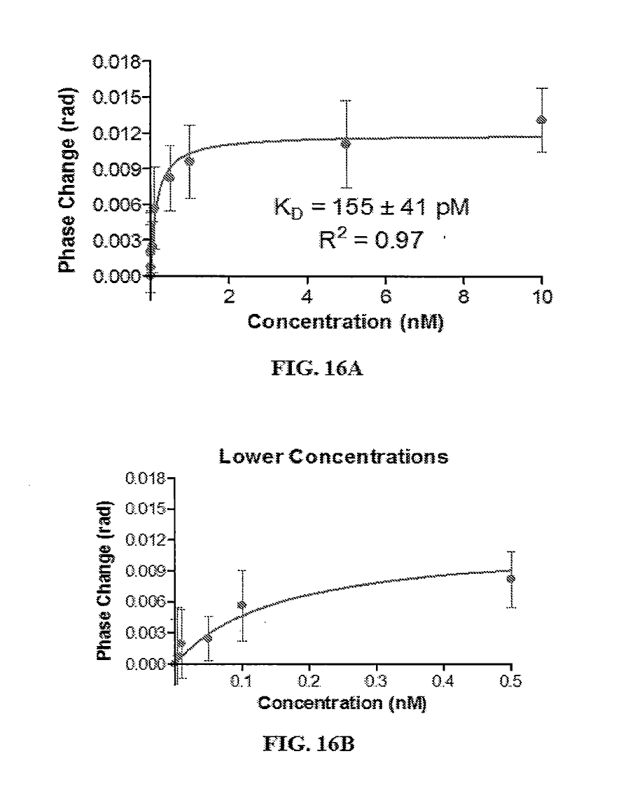

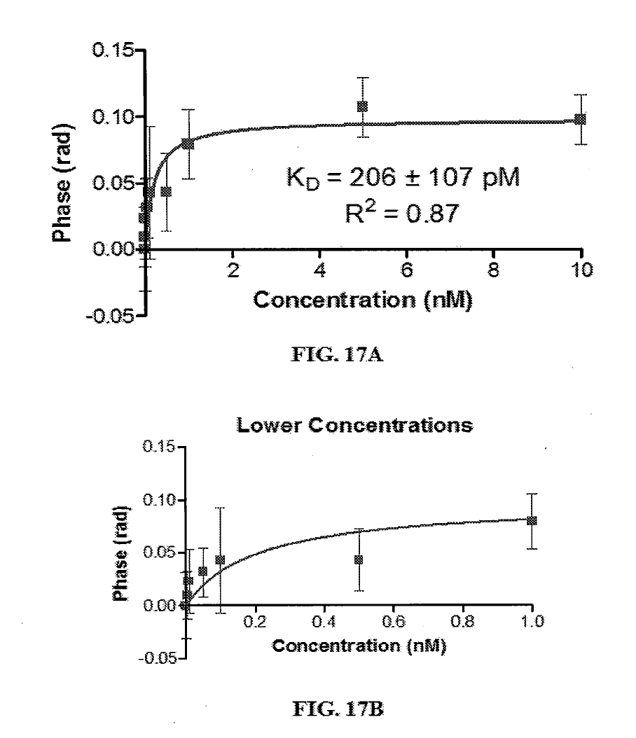

The experimental design is illustrated in FIG. 15. An affinity of 155±41 pM was measured using VRH in PBS, a value close to that measured in CHO cell vesicles (134 pM) (FIGS. 16A and 16B; see FIGS. 17A and 17B for binding affinity measured in serum). However, in this native environment the endogenously-expressed target concentration was 0.046 pM, now well below the K_{d} for the soluble form of the receptor. Following the theory as well as assay methodology for accurately determining binding affinity, this value is interpreted to be the “true” Kₐ for membrane-bound MAdCAM in this environment. What is again observed is a ˜20-fold decrease in binding affinity compared to soluble hMAdCAM. This result reinforces the observation that environmental restrictions change the binding affinity, supporting the hypothesis that the membrane matrix efficiently constrains conformational adaptations of the target. Without wishing to be bound by theory, these data suggest that the femtomolar sensitivity of this platform outshines any existing methodology because binding events can be quantified in targets at endogenously-expressed levels, and also in small volume (40 n1), sparing use of valuable tissue samples.

7. In Vitro Affinity of Anti-MAdCAM MAb Binding to MAdCAM in Interstitial-Like Homogenate (ILH)

Here, the physical and chemical properties of both the membrane-bound protein and the tissue microenvironment soluble protein were exploited. Binding isotherms were performed for tissue vesicles in another layer of complexity: an ILH. The ILH was generated from healthy human colon tissue by a “tissue elution” method previously described (Wiig, H. and Swartz, M. A. (2012) Phsyiol. Rev. 92, 1005-1060), by breaking the tissue into smaller pieces via homogenization with a Bullet Blender Storm® (Next Advance Inc.) in PBS with 1× protease inhibitor (Thermo Scientific) and no EDTA. The sample was centrifuged at 2000 g for ten minutes and the supernatant collected for analysis. This methodology (FIG. 11) further accounts for “background” binding events and for expression levels of membrane-bound protein as well as for soluble MAdCAM found in the target interstitial (Lowe, P. J., et al. (2009) Basic Clin Pharmacol Toxicol 106, 195-209). This provides a more physio-realistic affinity prediction.

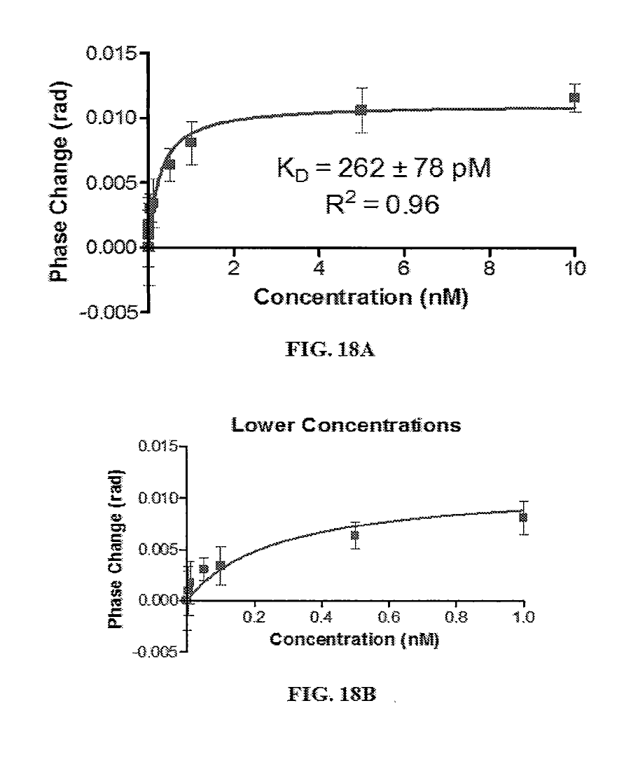

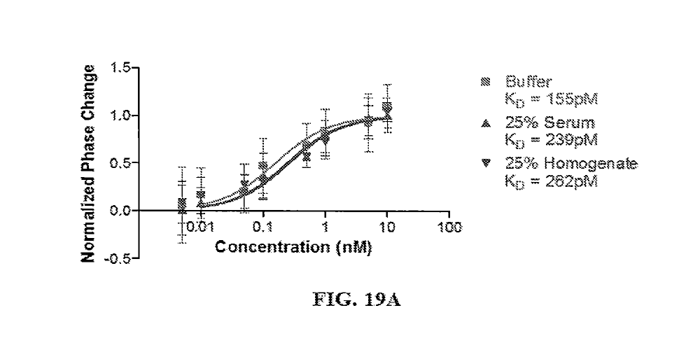

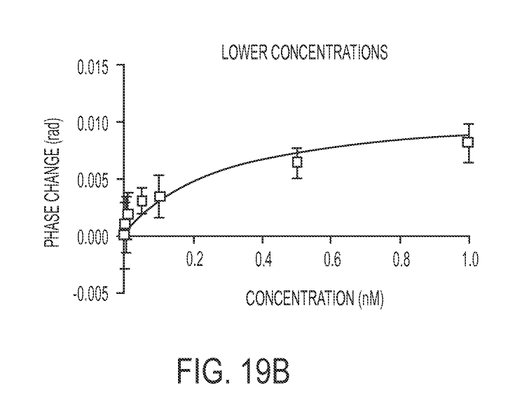

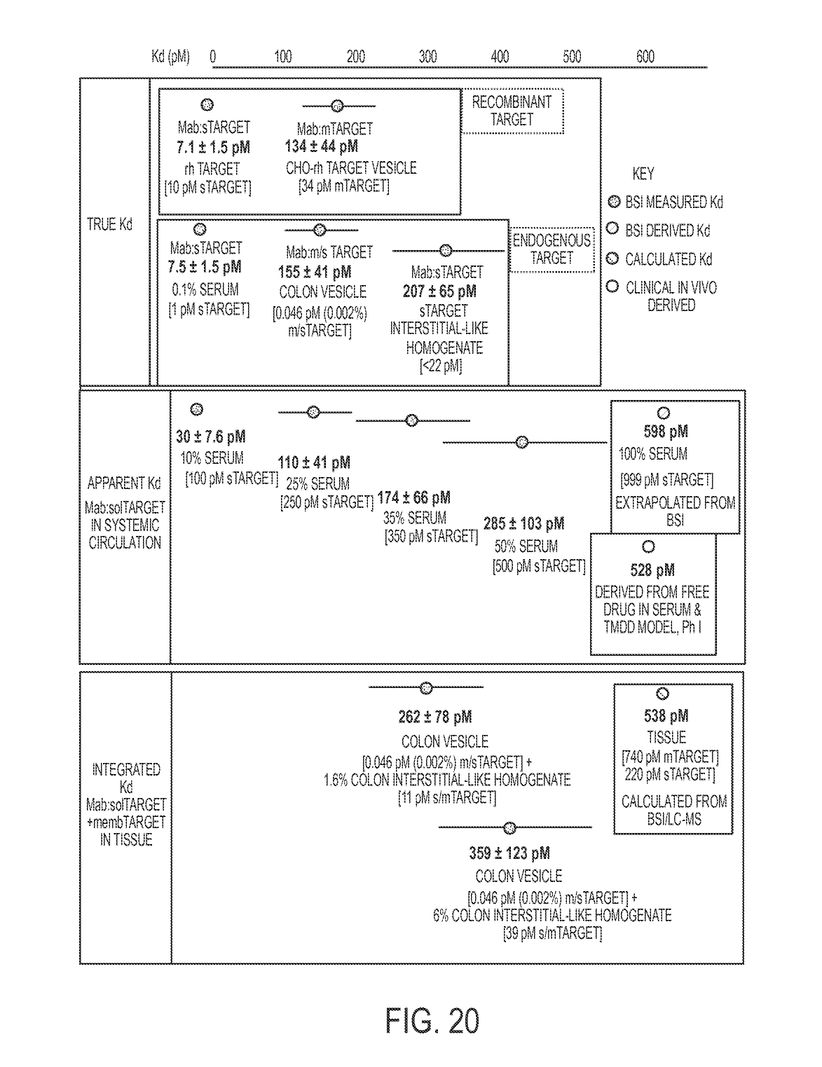

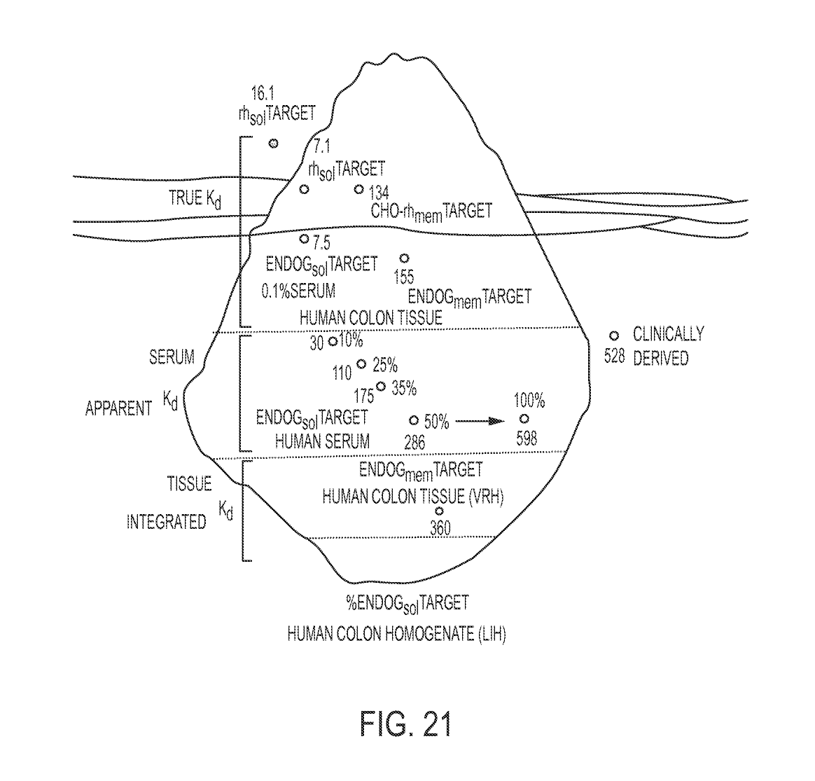

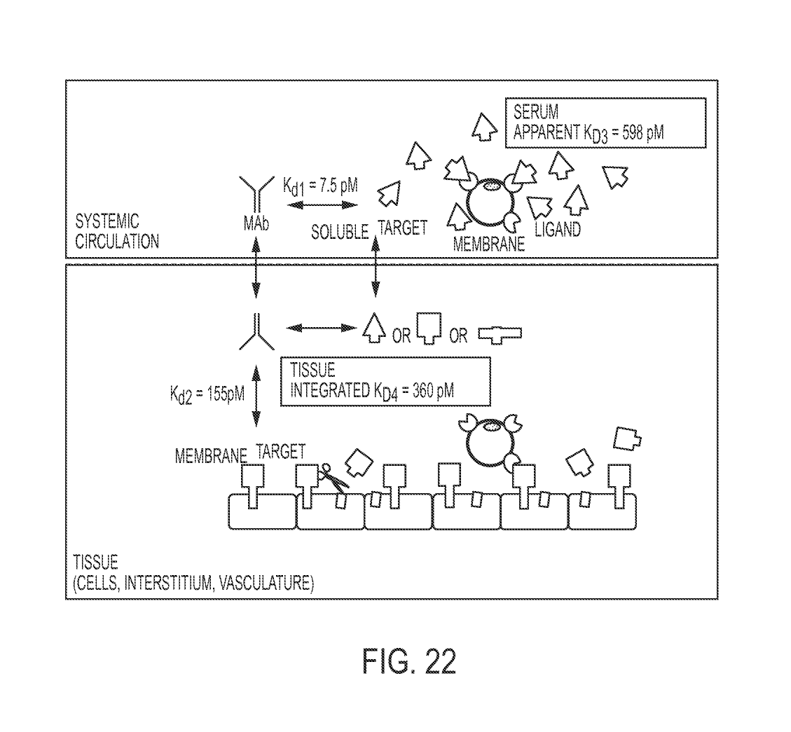

With vesicles bathed in 25% and 87.5% homogenate, an affinities of 262 (±78 pM) 360 (±123 pM) were measured, respectively (FIGS. 18A and 18B). Additionally, the ILH contained 11 pM (25%) and 39 pM (87.5%) of MAdCAM. Thus, the VRH is expressing endogenous levels of the target (0.046 mM) representing the cellular fraction, while the ILH at 87.5%, represents soluble MAdCAM target in the tissue space, making it the closest-to-physiological context that has ever been used in this type of assay. These values provide an eK_{d} that is proposed to be as close to a physiological value for 100% tissue that has been obtained by any in-vitro assay (FIGS. 19A and 19B). This ensemble narrates the story of how weaving together the anchoring environment with the presence of soluble target shifts the apparent affinity. This reflects the multiplicity of millieu effects, simulating drug diffusion and binding across matrices and allowing for ensemble tissue compartment measurements (eTCM) for eK_{d}.

8. In Vitro Affinity of MAb Binding

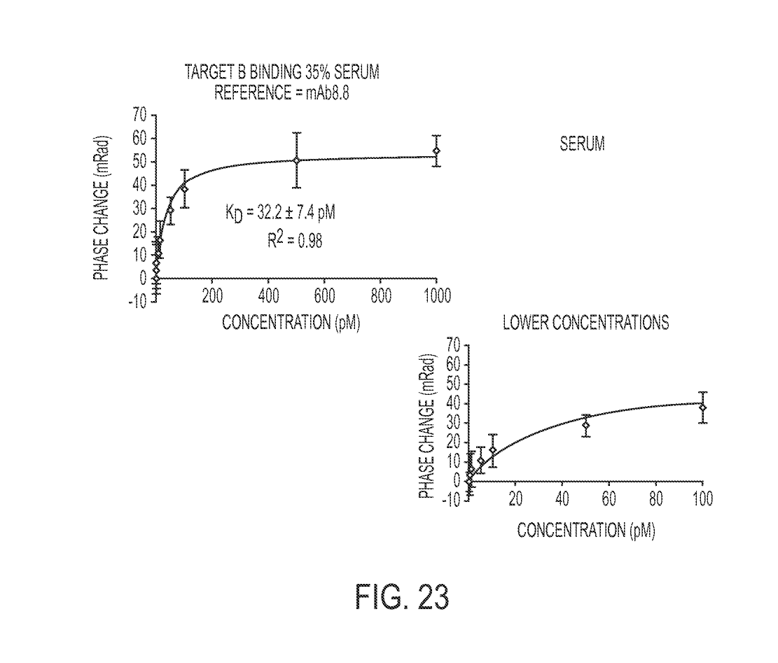

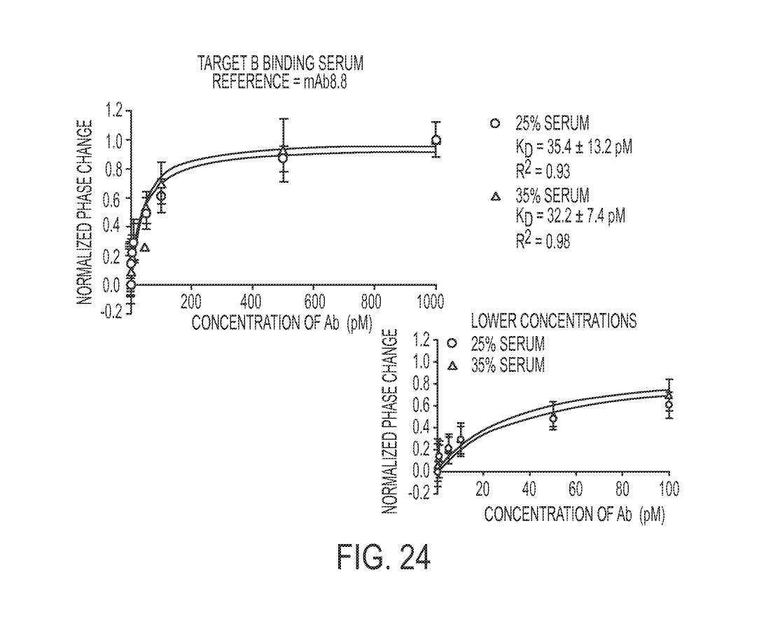

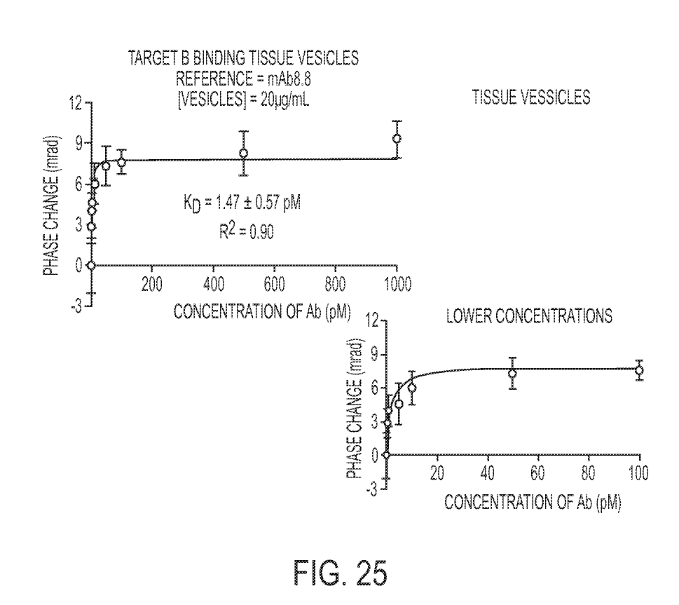

A second monoclonal antibody to a different (i.e., not related to MAdCAM) was used in BSI Kd assessments. This second antibody, referred to herein as “mAb B” or “target B mAb,” specifically binds a target (Target B) that is shed into the systemic circulation and is membrane-bound on PBMCs as well as intestinal tissue. This mAb was used to measure in vitro Kd values using BSI with 25% and 35% human normal serum resulting in a mean Kd of 34 pM (FIG. 23 and FIG. 24), which is in excellent agreement with the estimated clinically derived Kd of 40 pM. In addition, the Kd of mAb to membrane-bound target in normal human PBMC's and Chrohn's diseased human colon tissue the Kd of mAb to membrane-bound target is measured as 1.47+/−0.57 pM (FIG. 25).

9. Target B Serum Binding

Human serum was diluted in PBS to make a 50% serum solution. mAb B was diluted in PBS over a concentration range of 1 pM to 2 nM. mAb8.8 mAb, an isotype-matched negative control antibody known not to bind target B, was diluted in PBS over a concentration range of 1 pM to 2 nM. For the binding samples, the 50% serum solution was mixed 1:1 with the target B dilution series to result in a set of samples with 25% serum and a range of target B mAb from 0.5 pM to 1 nM. For the reference samples, the 50% serum solution was mixed 1:1 with the mAb8.8 dilution series to result in a set of samples with 25% serum and a range of mAb8.8 Ab from 0.5 pM to 1 nM. The samples were incubated at room temperature for 1 hour.

To measure the binding signal, the reference sample was injected into the channel and the BSI signal measured for 20 seconds. The channel was then evacuated and the binding sample with the same mAb concentration was injected into the channel and the BSI signal measured for 20 seconds. The channel was rinsed. The previous two steps were repeated for increasing concentrations of mAb. After the highest concentration of mAb (1 nM), the channel was thoroughly rinsed and steps 7-10 were repeated for three complete trials. The binding signal was calculated as the difference between the sample and reference signals for the same mAb concentration. This signal was plotted versus concentration and fitted with a single-site saturation binding curve to determine the affinity. See FIG. 23. The serum binding experiment was then repeated using the same protocol, except that the final concentration of serum was increased to 35% (initial dilution of serum in step 1 was 70%). See FIG. 24.

10. Target B Tissue Binding

Approximately 50 mg of human colon tissue was weighed out. The tissue sample was homogenized using a mortar and pestle. The homogenized tissue was suspended in 2 mL of PBS containing protease inhibitors. The solution was probe sonicated on ice for 2 minutes in a pulsed manner (5 seconds on, 1 second off). The solution was then centrifuged at 10,000 g at 4° C. for 1 hour. The supernatant was collected and DLS was done to measure size and polydispersity of the vesicles. If the polydispersity of the vesicles is >25%, then the solution was probe sonicated on ice for 90 seconds in a pulsed manner (5 seconds on, 1 second off). The solution was then centrifuged at 10,000 g at 4° C. for 1 hour. The supernatant was collected and DLS was done to measure size and polydispersity of the vesicles. The total protein concentration in the vesicle solution was measured using a Bradford assay.

The vesicle solution was diluted with PBS to make a 40 ng/mL total protein solution. Target B mAb was diluted in PBS over a concentration range of 1 pM to 2 nM. mAb8.8 Ab was diluted in PBS over a concentration range of 1 pM to 2 nM. For the binding samples, the 40 ng/mL total protein was mixed 1:1 with the Target B dilution series to result in a set of samples with 20 ng/mL total protein and a range of Target B Ab from 0.5 pM to 1 nM. For the reference samples, the 40 ng/mL total protein solution was mixed 1:1 with the mAb8.8 dilution series to result in a set of samples with 20 ng/mL total protein and a range of mAb8.8 Ab from 0.5 pM to 1 nM. The samples were incubated at room temperature for 1 hour.

To measure the binding signal, the reference sample was injected into the channel and the BSI signal measured for 20 seconds. The channel was then evacuated and the binding sample with the same Ab concentration was injected into the channel and the BSI signal measured for 20 seconds. The channel was rinsed. The previous two steps were repeated for increasing concentrations of Ab. After the highest concentration of Ab (1 nM), the channel was thoroughly rinsed and steps 7-10 were repeated for three complete trials.

The binding signal was calculated as the difference between the sample and reference signals for the same Ab concentration. This signal was plotted versus concentration and fitted with a single-site saturation binding curve to determine the affinity. See FIG. 25.

11. PBMC Vesicle Binding

A cell pellet containing roughly 5×10⁶ cells was resuspended in 1.5 mL of PBS containing protease inhibitors. The solution was probe sonicated on ice for 90 seconds in a pulsed manner (5 seconds on, 1 second off). The solution was then centrifuged at 10,000 g at 4° C. for 1 hour. The supernatant was collected and DLS was done to measure size and polydispersity of the vesicles. If the polydispersity of the vesicles is >25%, then the solution was probe sonicated on ice for 90 seconds in a pulsed manner (5 seconds on, 1 second off). The solution was then centrifuged at 10,000 g at 4° C. for 1 hour. The supernatant was collected and DLS was done to measure size and polydispersity of the vesicles. The total protein concentration in the vesicle solution was measured using a Bradford assay.

The vesicle solution was diluted with PBS to make a 40 μg/mL total protein solution. Target B mAb was diluted in PBS over a concentration range of 1 pM to 2 nM. mAb8.8 mAb was diluted in PBS over a concentration range of 1 pM to 2 nM. For the binding samples, the 40 μg/mL total protein was mixed 1:1 with the Target B dilution series to result in a set of samples with 20 μg/mL total protein and a range of Target B mAb from 0.5 pM to 1 nM. For the reference samples, the 40 μg/mL total protein solution was mixed 1:1 with the mAb8.8 dilution series to result in a set of samples with 20 μg/mL total protein and a range of mAb8.8 Ab from 0.5 pM to 1 nM. The samples were incubated at room temperature for 1 hour.

To measure the binding signal, the reference sample was injected into the channel and the BSI signal measured for 20 seconds. The channel was then evacuated and the binding sample with the same mAb concentration was injected into the channel and the BSI signal measured for 20 seconds. The channel was rinsed. The previous two steps were repeated for increasing concentrations of mAb. After the highest concentration of mAb (1 nM), the channel was thoroughly rinsed and steps 7-10 were repeated for three complete trials.

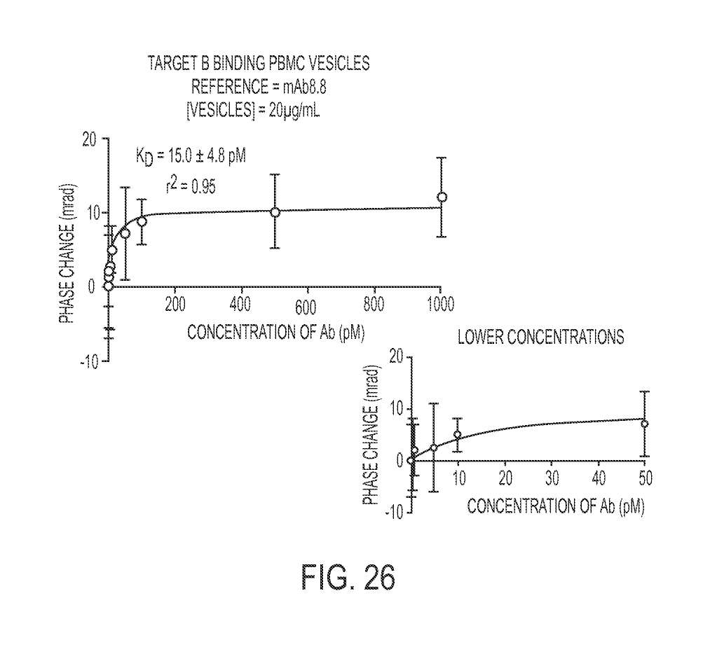

The binding signal was calculated as the difference between the sample and reference signals for the same mAb concentration. This signal was plotted versus concentration and fitted with a single-site saturation binding curve to determine the affinity. See FIG. 26.

12. PBMC Whole Cell Binding

A cell pellet containing roughly 5×10⁶ cells was resuspended in 1.5 mL of PBS. The total protein concentration in the vesicle solution was measured using a Bradford assay. The vesicle solution was diluted with PBS to make a 40 μg/mL total protein solution. Target B mAb was diluted in PBS over a concentration range of 1 pM to 2 nM. mAb8.8 Ab was diluted in PBS over a concentration range of 1 pM to 2 nM. For the binding samples, the 40 μg/mL total protein was mixed 1:1 with the Target B dilution series to result in a set of samples with 20 μg/mL total protein and a range of Target B mAb from 0.5 pM to 1 nM. For the reference samples, the 40 μg/mL total protein solution was mixed 1:1 with the mAb8.8 dilution series to result in a set of samples with 20 μg/mL total protein and a range of mAb8.8 Ab from 0.5 pM to 1 nM. The samples were incubated at room temperature for 1 hour.

To measure the binding signal, the reference sample was injected into the channel and the BSI signal measured for 20 seconds. The channel was then evacuated and the binding sample with the same mAb concentration was injected into the channel and the BSI signal measured for 20 seconds. The channel was rinsed. The previous two steps were repeated for increasing concentrations of mAb. After the highest concentration of mAb (1 nM), the channel was thoroughly rinsed and steps 7-10 were repeated for three complete trials.

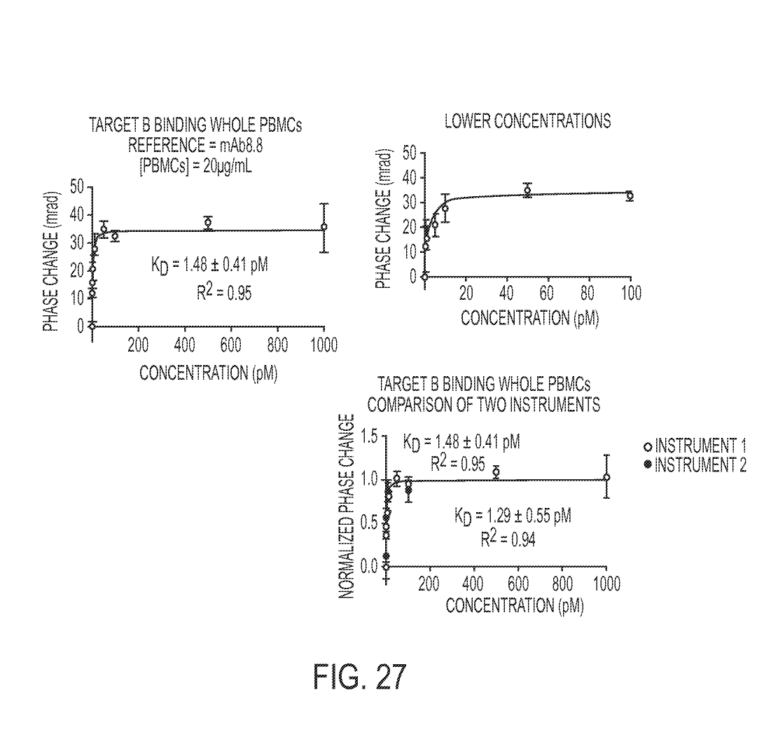

The binding signal was calculated as the difference between the sample and reference signals for the same mAb concentration. This signal was plotted versus concentration and fitted with a single-site saturation binding curve to determine the affinity. See FIG. 27.

For whole cells binding compared to vesicles binding for PBMCs with Target B Antibody, there is a notable difference in error bars and magnitude of the signal. The samples have not been modified. Even though receptor in native environment, in both cases, the cells exhibit a significant advantage.

13. Palbociclib Binding

The disclosed invention is not limited to antibody-protein interactions, but is applicable to a wide range of systems. The signal in BSI is generated by changes in RI of the solution when the binding partners undergo conformation and hydration changes upon binding. Since the magnitude of the BSI response is not mass dependent, as with most other label-free methods, small molecule-target (protein, DNA, RNA, etc.) interactions produce robust signals without amplification.

If there is a high-affinity ligand and a known receptor (target), as demonstrated here, an assay can be rapidly developed for use in tissues, serum, or other clinically relevant samples. Once the binding assay has been demonstrated, the small molecule can be used as the probe to quantify the presence of the receptor, monitor circulating concentrations of the receptor, and even evaluate efficacy of the therapy. A BSI assay is quantitative, requires no additional labeling or chemical modification, and directly represents the therapeutic system under investigation. Thus, Tissue-BSI automatically enables a companion diagnostic that can guide patient selection and stratification. In the case where the target receptor is indicative of disease state, the assay can be used as a diagnostic. If target coverage is important, yet the inhibitor has significant side effects, the assay can be used to optimize and monitor dose.

One example of using Tissue-BSI for detection of biological interactions with small molecules is the disclosed methods applied to cyclin-dependent Kinase 4/6 (CDK 4/6) inhibitor, palbociclib (IBRANCE):

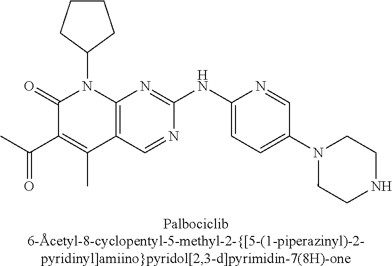 [SMILES: CC(=O)c1c(C)c2cnc(Nc3ccc(N4CCNCC4)cn3)nc2n(C2CCCC2)c1=O]

This kinase inhibitor is now approved for use in combination with letrozole for the treatment of postmenopausal women with estrogen receptor (ER)-positive, human epidermal growth factor receptor 2 (HER2)-negative advanced breast cancer as initial endocrine-based therapy for their metastatic disease. By simply performing a BSI-tissue assay on samples from perspective patients, it will be possible to; 1) determine suitability for the IBRANCE therapy, 2) monitor delivery using urine, serum or tissue samples, and 3) follow response to therapy.

As an example, breast tissue can be obtained (e.g., by biopsy) from a patient (e.g., an adult female diagnosed with an increased likelihood of breast cancer). The sample can be taken before therapy with palbociclib, during therapy with palbociclib, or after completion of therapy with palbociclib. The uncultured tissue can then be homogenized by blending, and the tissue homogenate can then be introduced into an instrument suitable for performing BSI analysis. Either before or after introducing the homogenate into the instrument, palbociclib is also introduced into the channel of the instrument and is allowed to interact with the tissue homogenate. Measurements similar to those described above can then be obtained, and the data can be plotted as shown in the Figures. Kd can then be determined.

### M. DISCUSSION

The current methods for measuring binding affinity of new drugs for their targets are typically reductionistic and time-consuming, requiring significant sample quantities and oftentimes providing dubious estimates for human dosing. Herein, comparable results to an existing method have been demonstrated, under a similar in vitro experimental condition (SPR). Further, the endogenous target was rapidly measured in native conditions, across increasing matrix complexity, using a single platform. The observed decrease in apparent drug affinity, from buffer to serum to tissue, in an increasingly indigenous target habitat, is a tangled web to unravel. Untangling this web is imperative for accurate prediction of safe and therapeutic dosing in humans.

At the surface, there appears to be a simple relationship between soluble receptor and the K_{d}. Upon moving from buffer to increasing amounts of soluble receptor in serum the apparent K_{d} was found to have an inverse, linear relationship with protein concentration (e.g., as target protein increases, affinity decreases). This well-behaved relationship allowed the extrapolation of a value that validated the modeled, predicted in vivo (clinically derived) value. Upon moving from serum to the “deeper” context of the native membrane environment, the true K_{d} was found to be quantifiably and notably shifted. Not unexpectedly, the binding affinity of the membrane-bound target is distinct and different from the circulating population. However, although this difference may have been surmised, being able to actually quantify this difference in affinity with changing environment cannot be understated and is unique to BSI assays described herein. To insure that no contributing factor slipped through this inclusive net, the interstitial domain was added and indicated that by accounting for binding events here, a reticulation of structures was encircled in a complete network, each part of which has a role in the harvest of data.

A physiologically relevant eK_{d} was measured that closely approximates the calculated in vivo (clinically derived) binding affinity. This is significant because it demonstrates that meaningful thermodynamic measurements for membrane-associated molecules that fully accounts for “off-site” drug binding are quite possible in a rapid, low volume format. Current methods, several of which must be employed, have only been the tip of the iceberg with regard to tapping into the potential for physiologic relevance. It is imperative to improve upon existing affinity modeling methods to offer better dose predictors for clinical efficacy and/or safety (Vermeire, S., et al. (2010) Gut 60, 1068-1075). Binding complexity and target dispensation are indicated as the effectors that largely matter for seeing below the surface of tethering and labeling of reagents in a non-native environment.

Herein binding experiments were performed under more natural and authentic conditions. It is predicted that eTCM will be a valuable tool for connecting the grid of interlacing biological fibers and unite pharmacology with human dosing regimes. During early drug discovery and prior to therapeutic candidate selection, potency measures made in relevant human tissue(s) enable real time adjustments of structure and affinity, likely reducing drug affinity optimization campaigns. In addition, clinical translation will likely move forward with greater confidence and less expense as a result of the teachings provided herein.

It will be apparent to those skilled in the art that various modifications and variations can be made in the present invention without departing from the scope or spirit of the invention. Other aspects of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method of detecting a binding interaction, the method comprising the steps of:
(a) preparing a sample comprising uncultured tissue homogenate;
(b) providing an apparatus adapted for performing light scattering interferometry, the apparatus comprising:
(i) a fluidic device;
(ii) a channel formed in the fluidic device capable of receiving the sample and an analyte;
(iii) a light source for generating a light beam;
(iv) a photodetector for receiving scattered light and generating intensity signals; and
(v) at least one signal analyzer capable of receiving the intensity signals and determining therefrom a binding interaction between the sample and the analyte;
(c) introducing the sample and the analyte into the channel; and
(d) interrogating the sample using light scattering interferometry.

2. The method of claim 1, wherein the binding interaction is between antibody-antigen, protein-protein, small molecule-small molecule, small molecule-protein, drug-receptor, enzyme-substrate, protein-DNA, protein-aptamer, DNA-DNA, RNA-RNA, DNA-RNA, protein-RNA, small molecule-nucleic acid, biomolecule-molecular imprint, biomolecule-carbohydrate, small molecule-membrane-bound protein, or antibody-membrane-bound protein.

3. The method of claim 1, wherein the tissue homogenate comprises at least one of a protein, small molecule, nucleic acid, polypeptide, carbohydrate, lipid, glycoprotein, lipoprotein, DNA, RNA, DNA-protein construct, or RNA-protein construct.

4. The method of claim 1, wherein the analyte comprises at least one of a small molecule, nucleic acid, polypeptide, carbohydrate, lipid, protein, glycoprotein, lipoprotein, DNA, RNA, DNA-protein construct, or RNA-protein construct.

5. The method of claim 1, wherein the sample and the analyte are introduced into the channel in label-free solution.

6. The method of claim 1, wherein the fluidic device and channel together comprise a capillary tube.

7. A method of detecting a binding interaction, the method comprising the steps of:
(a) preparing a sample comprising uncultured tissue homogenate;
(b) providing a fluidic device having a channel formed therein for reception of the sample and the analyte;
(c) introducing the sample and the analyte into the channel;
(d) directing a light beam from a light source onto the fluidic device such that the light beam is incident on at least a portion of the sample to generate scattered light through reflective and refractive interaction of the light beam with a fluidic device/channel interface, and the sample, wherein the scattered light comprising interference fringe patterns including a plurality of spaced light bands whose positions shift in response to changes in the refractive index of the sample;
(e) detecting positional shifts in the light bands; and
(f) determining the binding interaction between the sample and the analyte from the positional shifts of the light bands in the interference fringe patterns.

8. The method of claim 7, wherein the fluidic device and channel together comprise a capillary tube.

9. The method of claim 7, wherein the fluidic device comprises a silica substrate and an etched channel formed in the device for reception of the sample and/or analyte, the channel having a cross-sectional shape.

10. The method of claim 7, wherein the cross-sectional is semicircular.

11. A method of predicting the in vivo binding affinity of an analyte, the method comprising the steps of:
(a) preparing a sample comprising uncultured tissue homogenate;
(b) providing a fluidic device having a channel formed therein for reception of the sample and the analyte;
(c) introducing the sample and an analyte into the channel;
(d) directing a light beam from a light source onto the fluidic device such that the light beam is incident on at least a portion of the sample to generate scattered light through reflective and refractive interaction of the light beam with a fluidic device/channel interface, and the sample, wherein the scattered light comprising interference fringe patterns including a plurality of spaced light bands whose positions shift in response to changes in the refractive index of the sample;
(e) detecting positional shifts in the light bands;
(f) determining the K_{D} of the sample and the analyte using the positional shifts in the light bands; and
(g) predicting the in vivo behavior using the binding affinity.

12. The method of claim 11, wherein the analyte comprises at least one of a small molecule, nucleic acid, polypeptide, carbohydrate, lipid, protein, glycoprotein, lipoprotein, DNA, RNA, DNA-protein construct, or RNA-protein construct.

13. The method of claim 11, wherein the analyte comprises an antibody.

14. The method of claim 11, wherein the analyte comprises at least one small molecule.

15. The method of claim 14, wherein the small molecule is a drug candidate.
